(19) United States
(12) Patent Application Publication
(10) Pub. No.: US 2009/0214643 A1
(43) Pub. Date: Aug. 27, 2009

(54) IMPROVED PHARMACOKINETIC PROFILE OF BETA-ADRENERGIC INVERSE AGONISTS FOR THE TREATMENT OF PULMONARY AIRWAY DISEASES

(76) Inventors: Amie E. Franklin, Pacifica, CA (US); Michael Flashner, Foster City, CA (US); William J. Garner, San Francisco, CA (US); David Wurtman, San Diego, CA (US); Richard A. Bond, Houston, TX (US)

Correspondence Address: CATALYST LAW GROUP, APC, 9710 SCRANTON ROAD, SUITE S-170, SAN DIEGO, CA, 92121, US

(21) Appl. No.: 11/996,056

(22) PCT Filed: Jul. 19, 2006
(86) PCT No.: PCT/US06/27937

§ 371 Date: Nov. 7, 2008

Related U.S. Application Data
(60) Provisional application No. 60/701,304, filed on Jul. 19, 2005.

(51) Int. Cl. A61K 9/22; A61K 9/10; A61K 31/138; A61P 9/12

(52) U.S. Cl. 424/468; 424/484; 514/651

(57) ABSTRACT

Methods for administering beta-adrenergic inverse agonists with improved pharmacokinetic profiles for the treatment of pulmonary airway diseases are disclosed. The beta adrenergic inverse agonists are formulated in a controlled-release formulation comprising: (1) a beta-adrenergic inverse agonist in a therapeutically effective quantity; and (2) at least one agent that controls release of the beta-adrenergic inverse agonist resulting in a pharmacokinetic profile that minimizes acute detrimental reduction in airway function with the first dose and with each successive dose. This pharmacokinetic profile typically results in slow release of the drug into the bloodstream resulting in a large average Tₘₐₓ>4 hours. The controlled-release formulation may consist of multilayered matrix or erosional tablets, gastric-retention tablets, osmotic pump oral formulations, multiparticulate capsules or tablets or dermal patch.

### BACKGROUND OF THE INVENTION

The present invention relates to formulations and methods for administering beta-adrenergic inverse agonists with improved pharmacokinetic profiles for the treatment of pulmonary airway diseases such as asthma or chronic obstructive pulmonary diseases (COPD).

Beta-adrenergic inverse agonists are members of the general class of drugs called “beta blockers.” These drugs bind to inactive forms of beta-adrenergic receptors and prevent their activation from either endogenous agonists or exogenous agonists. This activity has been useful for the following disorders: hypertension, angina, glaucoma, congestive heart failure, tachycardia, and arrhythmia. In addition to these benefits, this class of drugs has demonstrated side-effects that have led them to be contra-indicated in patients with airway diseases, most notably asthma and chronic obstructive pulmonary disease (COPD). Beta-adrenergic receptors are located on the smooth muscle underlying the airways of the lungs and are responsible for ongoing bronchodilation. High level occupancy of these receptors prevents these receptors from activating bronchodilation. The net result would be bronchoconstriction or even bronchospasm, possibly due to unopposed airway constriction due to pulmonary muscarinic or other constricting receptors also located on the smooth muscle of the airways.

Patients with pulmonary airway diseases such as asthma or COPD already have impaired airway function and reducing their airway function even further may have serious effects resulting in an asthma exacerbation or ‘attack’ or may even lead to status asthmaticus, a severe and unopposed airway constriction that may lead to death if not aggressively managed in a hospital setting. Either knowingly or unknowingly, patients with asthma have taken beta-blockers for cardiovascular diseases and have had serious bronchoconstriction. A reduction in airway function in asthmatics was observed for the first approved beta blocker, pronethalol (Imperial Chemical Industries, Ltd. Alderlin Data for Clinical Investigators, 1962) and then for propranolol (R. S. McNeill Lancet 1101-1102 (1964)). The medical literature has many accounts of asthmatics having serious pulmonary function reduction in response to beta blocker administration including nadolol (Raine et al., Br. Med. J. 282: 548-549 (1981), metoprolol (Andersen et al., Br. J. Dis. Chest 73(4):407-8 (1979)), propranolol (Spitz, Am. J. Forensic Med. Pathol. 24(3):271-2 (2003)), timolol eye drops (Charan et al., Arch. Intern. Med. 140(6):843-4 (1980)), or sotalol (Devereux et al., Br. J. Clin. Pharmacol. 46(1)-79-82 (1998)). Consequently, administration of pharmacological doses of beta blockers has not been considered safe for patients with pulmonary airway diseases such as asthma and COPD and thus has been and continues to be contraindicated by government regulatory authorities.

In spite of this contraindication, Bond in PCT Patent Application Serial No. PCT/US2004/033157, filed Oct. 8, 2004, incorporated herein in its entirety by this reference, demonstrated that the subclass of beta-blockers, beta-inverse agonists, when dosed chronically to asthmatic mice was surprisingly able to protect their pulmonary airways from significant constriction in response to allergen and methacholine challenge. This benefit was as pronounced as a single acute dose of the beta agonist salbutamol which is used by many asthmatics for bronchodilation as a rescue medication during an asthma attack. However, a limitation of these experiments was that mice were administered the beta-adrenergic inverse agonists in the animal chow and the mice ate unknown amounts of the chow at unknown times. Additionally, observations of noticeable distress were noted in the mice in the first couple of days of drug treatment. It is thought that this distress was due to airway obstruction due to the drug treatment. Additionally, infusion experiments with beta-adrenergic inverse agonists directly into the mouse's bloodstream demonstrated that high levels of the drugs in the bloodstream led to severe bronchoconstriction similar to the bronchoconstriction observed with human adverse events to these drugs. These mouse studies demonstrated that beta-adrenergic inverse agonists in the acute term worsen airway function whereas chronic treatment dramatically improves airway function. The goal of treating humans is to capitalize on the chronic benefit of beta-adrenergic inverse agonists for pulmonary airway diseases while avoiding the acute detriment observed. Thus, the mouse studies do not provide guidance for human drug pharmacokinetics, i.e. the analysis of amount of drug in the bloodstream over time, during the early stages of treatment when the potential for asthma attacks are at their highest.

Another common problem with immediate release formulations containing beta-adrenergic inverse agonists is the consequence of rebound effects such as airway or cardiovascular symptoms upon abrupt cessation of therapy.

Consequently, there is a tremendous need for new therapeutic alternatives to β₂-adrenergic agonist use in asthmatics and in patients suffering from other pulmonary airway diseases and conditions modulated by beta₂-adrenergic receptors. Because these drugs are currently contraindicated in asthmatics, current formulations of drugs containing beta-adrenergic inverse agonists are not optimally formulated for safety and efficacy in patients with pulmonary airway diseases.

Therefore, there is a particular need for formulations that provide an improved pharmacokinetic profile of beta-adrenergic inverse agonists, particularly for the treatment of asthma and chronic obstructive pulmonary disease (COPD).

There is also a need for formulations that are formulated for the treatment of asthmatic conditions that are associated with particular times of day, such as nocturnal asthma. Nocturnal asthma has become recognized as a symptom that indicates an advanced disease process, and needs to be treated specifically.

### SUMMARY OF THE INVENTION

One aspect of the invention is a controlled-release formulation of an active beta-adrenergic inverse agonist comprising:

(1) an active beta-adrenergic inverse agonist in a therapeutically effective quantity; and

(2) at least one agent that controls release of the beta-adrenergic inverse agonist resulting in a pharmacokinetic profile that minimizes acute detrimental reduction in airway function with the first dose and with each successive dose, including larger doses, administered to a subject with a condition treatable by the administration of a beta-adrenergic inverse agonist.

Typically, the active beta-adrenergic inverse agonist is formulated as part of a formulation selected from the group consisting of: (1) an oral matrix controlled-release formulation; (2) an oral multilayered controlled-release tablet formulation; (3) an oral multiparticulate controlled-release formulation; (4) an oral osmotic controlled-release formulation; (5) an oral chewable controlled-release formulation; and (6) a dermal controlled-release patch formulation.

Typically, the active beta-adrenergic inverse agonist is selected from the group consisting of nadolol, bupranolol, butoxamine, carazolol, carvedilol, ICI-118,551, levobunolol, metoprolol, propranolol, sotalol, and timolol, and the salts, solvates, analogues, congeners, mimetics, bioisosteres, stereoisomers, hydrolysis products, metabolites, precursors, and prodrugs thereof. Preferably, the active beta-adrenergic inverse agonist is nadolol.

Another aspect of the invention is a method of treatment of a pulmonary airway disease comprising administering a controlled-release formulation according to the present invention in a therapeutically effective quantity to a subject suffering from pulmonary airway disease alone or together with one or more co-morbid diseases such as hypertension, congestive heart failure, superventricular tachycardia, migraine prophylaxis, arrhythmia, angina, or myocardial infarction.

Typically, the pulmonary airway disease is selected from the group consisting asthma, bronchiectasis, bronchitis, chronic obstructive pulmonary disease, Churg-Strauss syndrome, the pulmonary sequelae of cystic fibrosis, emphysema, allergic rhinitis, and pneumonia. A pulmonary airway disease of particular significance is asthma; formulations according to the present invention can be formulated to treat nocturnal asthma. The method can, alternatively, comprise the separate administration of a second drug for treatment of the pulmonary airway disease in a therapeutically effective quantity. The second drug can be selected from the group consisting of a β₂-selective adrenergic agonist, a steroid, an anticholinergic, a methylxanthine compound, an anti-IgE antibody, a leukotriene modifier, or a phosphodiesterase IV inhibitor.

### BRIEF DESCRIPTION OF THE DRAWINGS

The following invention will become better understood with reference to the specification, appended claims, and accompanying drawings, where:

FIG. 1A is a graph showing the effect of Corgar® (nadolol, 10 mg single dose, half of 20 mg tablet) on forced expiratory volume in one second, FEV₁, on subjects with asthma over 4 hours and on second Y axis predicted levels of nadolol (ng/ml) in bloodstream over 4 hours. FIG. 1B is the calculated change in FEV₁ for each subject compared to their peak FEV1 during the 4 hour time period and compared to their FEV₁ at time=0.

FIG. 2A is a graph showing that chronic use of Corgard® (nadolol) over 9 weeks by subjects results in an improvement in the airway hyperresponsiveness as measured by PC20 with methacholine, in 8 of 10 subjects.

FIG. 2B is a table showing that the individual doses relative to the PC20 doubling dose revealing a dose-response relationship between the amount of the drug and the improvement in airway hyperresponsiveness.

FIG. 3 is a graph showing the predicted pharmacokinetic profile of single dose of 10 mg (half of 20 mg tablet) of Corgard® (nadolol) in ng/ml over 24 hours. Nadolol single-dose pharmacokinetics exhibit dose proportionality between 2 to 80 mg dose range (e.g. pharmacokinetic curve is superimposable, as determined by Dreyfuss et al., J. Clin. Pharm. 19: 712-720 (1979)) (predicted profile, not based on actual results).

FIG. 4 is a graph showing the pharmacokinetic profile of single dose of 80 mg tablet of Corgard® (nadolol) in ng/ml over 24 hours. Peak nadolol serum levels are observed at 3.5 hours after dosing (Cₘₐₓ) over 24 hours. From ANDA 074229, Zenith Goldline, Pharmaceuticals, Inc.

FIG. 5 is an improved predicted pharmacokinetic profile for controlled-release nadolol (Tₘₐₓ=8 hours) (predicted profile, not based on actual results).

FIG. 6 is an improved predicted pharmacokinetic profile for controlled-release nadolol (Tₘₐₓ=12 hours) (predicted profile, not based on actual results).

FIG. 7 is an improved predicted pharmacokinetic profile for controlled-release nadolol (Tₘₐₓ=16 hours) (predicted profile, not based on actual results).

FIG. 8 is an improved predicted pharmacokinetic profile for controlled-release nadolol (Tₘₐₓ=24 hours) (predicted profile, not based on actual results).

FIG. 9 is an improved predicted pharmacokinetic profile for controlled-release nadolol for nocturnal asthma (predicted profile, not based on actual results).

FIG. 10 is improved predicted pharmacokinetic profile #2 for controlled-release nadolol for nocturnal asthma symptoms, particularly for symptoms from 2 a.m. to 6 a.m. (predicted profile, not based on actual results).

FIG. 11 is improved predicted pharmacokinetic for enteric-coated controlled-release nadolol (predicted profile, not based on actual results).

### DETAILED DESCRIPTION OF THE INVENTION

The present invention provides for an improved pharmacokinetic profile of beta-adrenergic inverse agonists such that airway function is not significantly reduced with the first dose, thereby allowing patients with pulmonary airway diseases to initiate chronic therapy more safely in spite of the known risk of bronchoconstriction and bronchospasm for these drugs in this group of patients. This pharmacokinetic profile is achieved by controlled release of the beta-adrenergic inverse agonists into the bloodstream resulting in a pharmacokinetic profile in which the average Tₘₐₓ>4 hours, and such that the drug bloodstream levels increase gradually over time during the time interval to Tₘₐₓ. Tₘₐₓ is the time at which the plasma concentration of the beta-adrenergic inverse agonist is at its maximum for each dose interval. Optionally, the Cₘₐₓ/AUC is reduced, a parameter that contributes to the pharmacokinetic properties of the formulation.

Previous results in animal models of asthma demonstrated that chronic dosing of beta inverse agonists was beneficial in reducing pulmonary airway constriction in response to allergen and methacholine challenge as recited in Bond in PCT Patent Application Serial No. PCT/US2004/033157, filed Oct. 8, 2004, incorporated herein in its entirety by this reference. However, animals can tolerate severe airway constriction as part of a controlled experiment whereas human asthmatics cannot be placed at significant risk by pharmacological treatment due to the first rule of medicine from Hippocrates, “Primum non nocere,” i.e. “first, do no harm”. The present invention is intended to allow asthma patients to benefit from chronic dosing of beta inverse agonists by improving the safety of the pharmacokinetic profile by preventing rapid and steep increases of the drug into the bloodstream following drug administration.

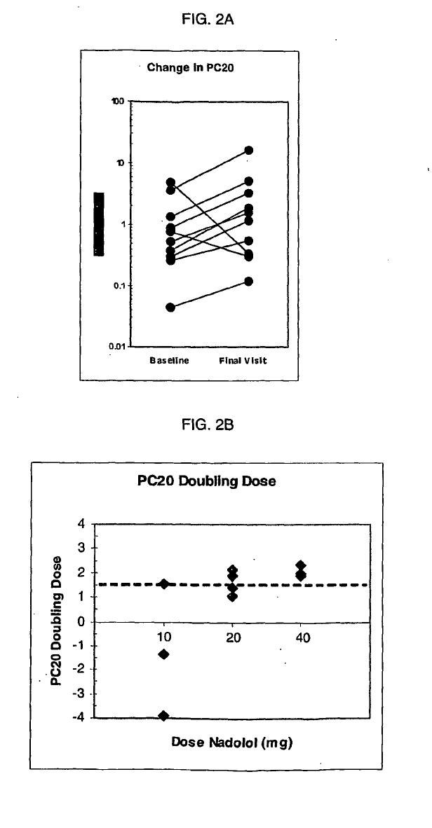

In the case of the preferred drug nadolol, the current pharmacokinetic profile of the approved drug and its generic counterparts (Corgard®, nadolol tablets, USP) results in a rapid rise of drug in the bloodstream to an average Tmax, about 3.5 hours, followed by a drop-off to approximately one-fourth of Tmax levels at 24 hours after dose administration based on single dose administration. The recommended dosing interval for Corgard®, nadolol tablets, USP is once a day (Corgard product Insert, Monarch Pharmaceuticals, Inc.). It has been discovered that when asthmatics were administered their first 10 mg dose of Corgard this resulted in a measurable reduction in pulmonary airway function as determined by measuring FEV₁ (forced expiratory volume in one minute) during the first four hours of dosing coincident with the rise to peak drug concentration—the average Tₘₐₓ of Corgard (time to maximum drug concentration in the bloodstream) is 3.5 hours. For 10 patients, the average drop in FEV₁ was 7.7% at the end of 4 hours. However, 2 subjects were observed to have a drop of 18% of FEV₁ which is clinically significant. As both of these subjects were mild asthmatics with an FEV₁>80% predicted, they were able to tolerate this dose in the clinic and did not require any intervention or rescue medication. However, for moderate or severe asthmatics an 18% drop in their FEV₁ would be sufficient to cause them to require airway support, for example a subject starting off with 60% FEV₁ predicted would have their airway below 50% FEV₁. Even for a mild asthmatic, a physician would be reluctant to routinely prescribe this drug in light of the acute detriment as even mild patients may experience a severe asthma exacerbation requiring emergency department visit, hospitalization, or steroid treatments (Zeiger et al., MIAMI Study Research Group. Variability of symptoms in mild persistent asthma: baseline data from the MIAMI study. Respir Med. 2004 September; 98(9):898-905). However, once the subjects tolerate these doses, despite their pharmacokinetic limitation, chronic dosing was observed to reduce airway hyperresponsiveness in 8 of 10 treated subjects as shown in FIG. 2A.

Once daily administration is a useful feature and helps to ensure patient compliance, however the pharmacokinetic profile of the approved formulation reduces airway function in patients with asthma and thus poses a potential safety risk upon first dose administration and with successive doses, including dose escalations. Consequently, it is non-obvious to develop a new once daily controlled-release formulation of the preferred drug nadolol when there is already an approved once-daily formulation for nadolol. However, as mentioned previously, the rationale for this is that the pharmacokinetic profile of the current once daily nadolol formulation results in maximal levels of drug in the bloodstream at an average of 3.5 hours after administering the first dose. This profile has been demonstrated to result in measurable airway constriction in mild asthmatics in a clinical study. However, this problem with the use of once-daily nadolol formulations is overcome by the formulations and methods of the present invention.

The pharmacokinetic profile is defined by a number of parameters. As used herein, the term “Tₘₐₓ” is defined as the time when the maximum concentration of the beta-adrenergic agonist is reached in the blood after administration of a formulation of the beta-adrenergic inverse agonist. Typically, what is referred to is the average Tₘₐₓ, which is defined herein as the statistical arithmetic mean across the patient population. As used herein, the term “Cₘₐₓ” is defined as the maximum concentration in the bloodstream of the beta-adrenergic inverse agonist reached after administration of a formulation of the beta-adrenergic inverse agonist, usually for one specified dosage level. Similarly, the term “Cₘᵢₙ” is defined as the minimum concentration of the beta-adrenergic inverse agonist in the bloodstream over a specified period. The term “half-life” is defined as the time to when there is half the Cₘₐₓ concentration of the beta-adrenergic inverse agonist in the bloodstream; this is affected by the rates of excretion and metabolism of the drug. The term “elimination rate constant” is defined as the rate constant of elimination of the beta-adrenergic inverse agonist via excretion and metabolism. The term “apparent volume distribution,” V, is defined as the apparent volume that the beta-adrenergic inverse agonist is distributed in, such as blood, or other compartments such as fat; this volume need not correspond to an actually-measurable physiological volume. The term “AUC,” area under the curve, is defined as the area under the plasma (serum, or blood) concentration of the beta-adrenergic inverse agonist versus time curve, as is conventionally determined in pharmacokinetics. With both single and multiple dosing, there is the peak to trough ratio where the peak is the Cₘₐₓ, and the trough is the Cₘᵢₙ. Pharmacokinetic parameters of a drug dosage form are required by regulatory bodies (e.g. FDA) to evaluate dosing regime in subjects and also special subjects with modified pharmacokinetic parameters such as reduced excretion due to renal failure. Pharmacokinetic parameters are additionally used to determine bioequivalence of drugs. Consequently, it is the pharmacokinetic profile that is particularly meaningful as this dictates the pharmacological effect over time of the drug.

As used herein, in the generally accepted two-state model of receptor theory, the term “agonist” is defined as a substance that has an affinity for the active site of a receptor and thereby preferentially stabilizes the active state of the receptor, or a substance, including, but not limited to, drugs, hormones, or neurotransmitters, that produces activation of receptors and enhances signaling by those receptors. Irrespective of the mechanism or mechanisms of action, an agonist produces activation of receptors and enhances signaling by those receptors.

As used herein, in the two-state model of receptor theory, the term “antagonist” is defined as a substance that does not preferentially stabilize either form of the receptor, active, or inactive, or a substance, including, but not limited to, drugs, hormones, and neurotransmitters, that prevents or hinders the effects of agonists and/or inverse agonists. Irrespective of the mechanism or mechanisms of action, an antagonist prevents or hinders the effects of agonists and/or inverse agonists.

As used herein, in the two-state model of receptor theory, the term “inverse agonist” is defined as a substance that has an affinity for the inactive state of a receptor and thereby preferentially stabilizes the inactive state of the receptor, or a substance, including, but not limited to, drugs, hormones, or neurotransmitters, that produces inactivation of receptors and/or prevents or hinders activation by agonists, thereby reducing signaling from those receptors.

The term “subject,” as used herein, refers to human or animal species. In general, methods and compositions according to the present invention can be used to treat not only humans, but also socially or economically important animal species such as cows, horses, sheep, pigs, goats, dogs, and cats. Unless specified, methods and compositions according to the present invention are not limited to treatment of humans.

The term “therapeutically effective amount,” as used herein, refers to an amount of a therapeutic agent or composition effective to treat, ameliorate, or prevent a desired disease or condition, or to exhibit a detectable therapeutic or preventative effect. The effect can be detected by, for example, chemical markers, antigen levels, or changes in physiological indicators such as airway resistance. Therapeutic effects also include reduction in physical symptoms, such as decreased bronchoconstriction or decreased airway resistance, and can include subjective improvements in well-being noted by the subjects or their caregivers. The precise therapeutically effective amount for a subject will depend upon the subject's size, weight, and health, the nature and extent of the condition affecting the subject, and the therapeutics or combination of therapeutics selected for administration, as well as variables such as liver and kidney function that affect the pharmacokinetics of administered therapeutics. Thus, it is not useful to specify an exact effective amount in advance. However, the effective amount for a given situation can be determined by routine experimentation and is within the judgment of the clinician. This is further described below.

As used herein, the term “controlled release” or equivalent terminology, such as “an agent that controls release,” includes all means and/or methods that delay release of an active agent, such as an active beta-adrenergic agonist, beyond the release time that would be observed if the agent were directly administered, typically orally, to a subject without the use of any such means or methods. In some alternatives, described below, the effect of the agent that controls release is to provide a gradual release of the active beta-adrenergic inverse agonist such that the inverse agonist is released into the bloodstream only gradually with a relatively extended period during which the concentration of the inverse agonist in the bloodstream increases. In other alternatives, also described below, the effect of the agent that controls release is to delay the onset of any release of the inverse agonist into the bloodstream for a defined period of time, after which the inverse agonist is released relatively rapidly into the bloodstream such that the concentration of the inverse agonist in the bloodstream rises at essentially the same rate as it would if the agent were administered without an agent to control release, beginning at the delayed onset of release. This second set of alternatives includes, but is not limited to, alternatives employing enteric coatings as described below.

Accordingly, one aspect of the invention is a formulation of an active beta-adrenergic inverse agonist that comprises: (1) an active beta-adrenergic inverse agonist in a therapeutically effective quantity; and (2) at least one agent that controls release of the beta-adrenergic inverse agonist resulting in a pharmacokinetic profile that minimizes acute detrimental reduction in airway function with the first dose and with each successive dose administered to a subject with a condition treatable by the administration of a beta-adrenergic inverse agonist. Typically, this pharmacokinetic profile results in slow release of the drug into the bloodstream, resulting in an average Tₘₐₓ of >4 hours than would occur by administration of an immediate-release formulation of the same active beta-adrenergic inverse agonist. Optionally, the Cₘₐₓ/AUC is reduced which contributes to smoothing the level of the drug over time.

These formulations are particularly valuable in the treatment of chronic conditions affecting the airway, as described below, particularly for the treatment of asthma.

The pharmacokinetic profile of the beta-adrenergic inverse agonist for the treatment of pulmonary airway disease can be achieved by administering a controlled-release formulation in which the active beta-adrenergic inverse agonist is formulated as part of: (1) an oral matrix controlled-release formulation; (2) an oral multilayered controlled-release tablet formulation; (3) an oral multiparticulate controlled-release formulation; (4) an oral osmotic controlled-release formulation; (5) an oral chewable controlled-release formulation; or (6) a dermal controlled-release patch formulation. Other alternatives are possible.

In the case of an oral matrix controlled-release formulation, the beta-adrenergic inverse agonist can be incorporated into a matrix such as those described in PCT Patent Publication No. WO 02/067905 by Gutierrez-Rocca or PCT Patent Publication No. WO 02/085112 by Gutierrez-Rocca et. al., both of which are incorporated herein by this reference. In general, in one alternative, the matrix can comprise a polymer mixture comprising a first component of about 80 weight percent polyvinylacetate combined with about 20 weight percent polyvinylpyrrolidone, combined with a second component of a cellulose ether polymer. In this alternative, the polymer mixture comprises the first component in an amount ranging from about 30 weight percent to about 80 weight percent of the total weight of the formulation and the second component comprises from about 40 weight percent to about 2 weight percent of the total weight of the formulation, the remainder being the active beta-adrenergic inverse agonist as described below. The cellulose ether polymer can be a methylcellulose polymer or a hydroxypropoxyl methylcellulose polymer.

In another alternative, the formulation can comprise a layer comprising a superporous hydrogel containing a functional group having a surface to which the active beta-adrenergic inverse agonist is associated to form at least one associated surface. The superporous hydrogel is formed by polymerization of one or more monomers selected from the group consisting of acrylic acid, acrylamide, vinylpyrrolidone, sulfopropyl acetate, hydroxyethyl methacrylate, hydroxypropyl methacrylate, and hydroxyethyl acetate. The monomer can be combined with a suitable cross-linker; the cross-linker can be selected from the group consisting of N′-methylene-bis-acrylamide, polyethylene glycol diacrylate, diethylene glycol diacrylate, and divinyl glycol. Other polymerization additives can be employed. The association between the active beta-adrenergic agonist and the hydrogel can be by hydrogen bonding, salt links (ionic bonds), covalent bonding, hydrophobic interactions, van der Waals forces, or other forces.

In the case of an oral multilayered controlled-release tablet formulation, the active beta-adrenergic inverse agonist can be incorporated into a formulation such as those described in U.S. Pat. No. 6,635,280 to Shell et al., U.S. Pat. No. 6,340,475 to Shell et al., U.S. Pat. No. 5,582,837 to Shell, or U.S. Pat. No. 5,007,790 to Shell, all of which are incorporated herein by this reference. In general, this dosage form comprises a plurality of solid particles of initially about 3-9 mm in diameter in maximum dimension, each particle containing a solid-state active beta-adrenergic inverse agonist dispersed within a non-chemically crosslinked alkyl-substituted cellulose selected from the group consisting of hydroxyethylcellulose, hydroxypropylcellulose, hydroxypropylmethylcellulose, carboxymethylcellulose, and hydroxymethylcellulose, in a weight ratio of active beta-adrenergic inverse agonist to polymer of about 1:9 to 9:1, the particles (1) swelling unrestrained dimensionally up to about three times their original diameter via imbibition of water from gastric fluid to increase the size of the particles to promote retention within the stomach, and to make the particles slippery, which also promotes their retention within the stomach, (2) permitting dissolution of the dispersed active beta-adrenergic inverse agonist by imbibed gastric water while the active beta-adrenergic inverse agonist is within the particle and release of the resulting solution, thus assuring that only active beta-adrenergic inverse agonist in solution contacts the gastric mucosa, (3) protecting undissolved active beta-adrenergic inverse agonist from stomach enzymes or pH and duodenum, and (4) maintaining their physical integrity over at least a substantial portion of the time period during which the active beta-adrenergic inverse agonist is released into the stomach and then dissolves; and wherein the dosage form is in the form of a tablet or capsule that maintains the particles in a packed mass prior to their ingestion and then rapidly disintegrates in the gastric fluid to permit the particles to disperse in the stomach.

In the case of the oral multiparticulate controlled-release formulation, the beta-adrenergic inverse agonist can be incorporated into a formulation such as those described in U.S. Pat. No. 6,663,888 to Percel et al., incorporated herein by this reference, in which a bimodal release profile is obtained that comprises: (1) immediate release (IR) beads comprising a core particle containing active beta-adrenergic inverse agonist; and (2) timed pulsatile release (TPR) beads, wherein the TPR beads comprise: (a) a core particle containing active beta-adrenergic inverse agonist; and (b) a pulse coating comprising a water insoluble polymer and an enteric polymer surrounding the core, the timed pulsatile release (TPR) beads when tested in a USP Type II apparatus at 50 rpm using a 2-stage dissolution medium (first 2 hours in 700 ml 0.1 N HCl at 37° C. followed by a dissolution in a pH of 6.8 obtained by the addition of 200 ml of pH modifier) exhibit a dissolution profile substantially corresponding to the following pattern: after 2 hours, 0-25% of the total active beta-adrenergic inverse agonist is released; after 3 hours, 15-80% of the total active beta-adrenergic inverse agonist is released; and after 4 hours, not less than 60% of the total active beta-adrenergic inverse agonist is released, wherein the IR beads provide a therapeutically effective amount of active beta-adrenergic inverse agonist and the TPR beads provide a delayed dose of active beta-adrenergic inverse agonist which provides a therapeutically effective amount of active beta-adrenergic inverse agonist. Typically, in this alternative, the enteric polymer is selected from the group consisting of esters of cellulose, polyvinyl acetate phthalate, pH-sensitive methacrylic acid-methylmethacrylate copolymers, shellac and derivatives thereof. More typically, the enteric polymer is selected from the group consisting of cellulose acetate phthalate, hydroxypropyl methylcellulose phthalate, hydroxypropyl methylcellulose succinate and combinations thereof. At least one of the polymers can further comprise a plasticizer. The plasticizer can be selected from the group consisting of triacetin, tributyl citrate, tri-ethyl citrate, acetyl tri-n-butyl citrate, diethyl phthalate, dibutyl sebacate, polyethylene glycol, polypropylene glycol, castor oil and acetylated mono- and di-glycerides and mixtures thereof.

In another alternative, the active beta-adrenergic inverse agonist can be incorporated into a formulation such as those described in U.S. Pat. No. 6,627,223 to Percel et al., incorporated herein by this reference, in which the formulation comprises a plurality of core particles, each core particle containing an active beta-adrenergic inverse agonist; the particle being coated with a first membrane of an enteric polymer; and a second membrane of a combination of a water-insoluble polymer and an enteric polymer wherein the water-insoluble and the enteric polymers are present in the second membrane at a weight ratio of about 10:1 to 1:1, and the total weight of the first and second coatings is about 15 to 80 weight percent based on the total weight of the coated particles; wherein the first and second membranes can be coated on the core particle in either order. In this alternative, the core particle can be a non-pareil sugar seed coated with a drug and polymeric binder; alternatively, the core particle is a particle prepared by granulation and milling or by extrusion/spheronization to form an active beta-adrenergic inverse agonist particle. The enteric polymer can be selected from the group consisting of esters of cellulose, polyvinyl acetate phthalate, pH sensitive methacrylic-methylmethacrylate copolymers and shellac. The water insoluble polymer of the second coating is selected from the group consisting of ethylcellulose, polyvinyl acetate, neutral copolymers based on ethyl acrylate and methylmethacrylate and copolymers of acrylic and methacrylic acid esters having quaternary ammonium groups. At least one of the membranes can further comprise a plasticizer, which can be selected from the group consisting of triacetin, tri-butyl citrate, tri-ethyl citrate, acetyl tri-n-butyl citrate, diethyl phthalate, castor oil, dibutyl sebacate, acetylated monoglycerides and mixtures thereof.

Still other alternatives are disclosed in U.S. Pat. No. 6,500,454 to Percel et al., U.S. Pat. No. 5,900,252 to Calanchi et al., and U.S. Pat. No. 5,047,248 to Calanchi et al., all of which are incorporated herein in their entirety by this reference. In general, according to these alternatives, the formulation can comprise a retarding base or matrix consisting of a polysaccharide of natural origin, alone or mixed with one or more natural or synthetic polymers which modify the release pattern so as to obtain a therapeutically effective formulation for the active beta-adrenergic inverse agonist.

Still other alternatives are disclosed in U.S. Pat. No. 6,814,979 to Rudnic et al., incorporated herein by this reference in this alternative, the active beta-adrenergic inverse agonist is incorporated into an osmotic pharmaceutical delivery system comprising (1) a semipermeable wall that maintains its integrity during pharmaceutical delivery and which has at least one passage through; and (2) a composition within the wall, the composition comprising: (a) the active beta-adrenergic inverse agonist; (b) at least one non swelling solubilizing agent which enhances the solubility of the active beta-adrenergic inverse agonist; (c) at least one non-swelling osmotic agent, and (d) at least one lubricant, the composition within the wall excluding a polymer that swells. The at least one non-swelling solubilizing agent can be selected from (A) agents that inhibit crystal formation of the pharmaceutical agent or acts by complexation therewith; (B) high HLB (hydrophilic-lipophilic balance) surfactants; (C) citrate esters; and (D) stearate salts; and combinations thereof. The at least one agent that inhibits crystal formation of the active beta-adrenergic inverse agonist can be selected from the group consisting of polyvinylpyrrolidone, polyethylene glycol, cyclodextrins, gelatin, maltodextrin, sorbitol, and polyglycenyl mixed vegetable fatty acid esters.

In yet another alternative, the active beta-adrenergic inverse agonist can be incorporated into an osmotic system such as that disclosed in U.S. Pat. No. 6,838,093 to Flanner et al., hereby incorporated by this reference. In general, the osmotic system comprises: (1) a core portion, wherein the core portion includes the active beta-adrenergic inverse agonist at a first concentration; (2) a layer portion enclosing and directly adjacent to the core portion, wherein the layer portion includes the active beta-adrenergic inverse agonist at a second concentration, the second concentration being greater than said first concentration; and (3) a semipermeable wall portion enclosing the core portion and the layer portion. The semipermeable wall portion can be formed from cellulose acetate.

In additional alternatives, the active beta-adrenergic inverse agonist can be incorporated into a sustained release formulation such as those disclosed in U.S. Pat. No. 6,811,794 to Burnside et al., U.S. Pat. No. 6,605,300 to Burnside et al., U.S. Pat. No. 6,514,532 to Rudnic et al., U.S. Pat. No. 5,952,004 to Rudnic et al., U.S. Pat. No. 5,883,103 to Burnside et al., or U.S. Pat. No. 5,824,638 to Burnside et al., all of which are incorporated herein by this reference. For example, the active beta-adrenergic inverse agonist can be incorporated into a formulation comprising a stable, hydrophobic emulsion comprising a continuous phase of a hydrophobic material selected from the group consisting of a long chain carboxylic acid or ester or alcohol thereof dispersed in an aqueous phase or having a hydrophilic discontinuous phase dispersed in a hydrophobic phase of a long chain carboxylic acid or alcohol thereof. The emulsion with the active beta-adrenergic inverse agonist is incorporated into a pharmaceutical carrier suitable for oral delivery.

In still another alternative for an oral multiparticulate controlled-release formulation, the active beta-adrenergic inverse agonist can be incorporated into a formulation such as the formulation of U.S. Pat. No. 6,902,742 to Devane et al., incorporated herein by this reference, which is a multiparticulate modified release composition that in operation delivers the active beta-adrenergic inverse agonist in a pulsed or bimodal manner. The multiparticulate modified release composition comprises an immediate release component and a modified release component; the immediate release component comprising a first population of active beta-adrenergic inverse agonist containing particles and the modified release component comprising a second population of the active beta-adrenergic inverse agonist containing particles coated with a controlled release coating; wherein the combination of the immediate release and modified release components in operation delivers the active ingredient in a pulsed or a bimodal manner.

In still another alternative for an oral multiparticulate controlled-release formulation, the active beta-adrenergic inverse agonist can be incorporated into a formulation such as the formulation of U.S. Pat. No. 6,399,100 to Clancy et al., U.S. Pat. No. 6,066,339 to Stark et al., U.S. Pat. No. 5,637,320 to Bourke et al., U.S. Pat. No. 5,616,345 to Geoghegan et al., U.S. Pat. No. 5,505,962 to Sparks, U.S. Pat. No. 5,354,556 to Sparks et al., U.S. Pat. No. 5,128,142 to Mulligan et al., U.S. Pat. No. 4,973,469 to Mulligan et al., U.S. Pat. No. 4,863,742 to Panoz et al., U.S. Pat. No. 4,826,688 to Panoz et al., U.S. Pat. No. 4,721,619 to Panoz et al., U.S. Pat. No. 4,716,040 to Panoz et al., or U.S. Pat. No. 4,663,150 to Panoz et al., all of which are incorporated herein by this reference. For example, the active beta-adrenergic inverse agonist can be incorporated into a multiparticulate formulation including a rate-controlling polymer such as a hydroxypropylmethylcellulose (HPMC) polymer, a hydroxypropylcellulose (HPC) polymer, a poly(ethylene oxide) polymer, an ethylcellulose polymer or a combination thereof present in an amount of 5 to 75% by weight, more preferably 20 to 50% by weight, most preferably 30 to 45% by weight in the preparation, as described in U.S. Pat. No. 6,399,100 to Clancy et al. In another example, the active beta-adrenergic inverse agonist is formulated in multi-particulate pellet form, each pellet having a core of the active beta-adrenergic inverse agonist in association with an organic acid, the active beta-adrenergic inverse agonist and the organic acid being present in a ratio of from 20:1 to 1:1, and a multi-layer membrane surrounding said core and containing a pharmaceutically acceptable film-forming, water insoluble polymer and optionally a pharmaceutically acceptable film-forming, water soluble polymer, as described in U.S. Pat. No. 5,637,320 to Bourke et al.

Other alternatives employ the devices of U.S. Pat. No. 5,997,501 to Gross et al., incorporated herein by this reference.

In the case of an oral osmotic controlled-release formulation, the active beta-adrenergic inverse agonist can be incorporated into a formulation such as the formulation disclosed in U.S. Pat. No. 6,840,931 to Peterson et al., U.S. Pat. No. 6,764,697 to Jao et al., U.S. Pat. No. 6,551,613 to Dong et al., U.S. Pat. No. 6,548,083 to Wong et al., 6,534,089 to Ayer et al., U.S. Pat. No. 6,524,305 to Peterson et al., U.S. Pat. No. 6,387,403 to Seroff et al., U.S. Pat. No. 6,368,626 to Bhatt et al., U.S. Pat. No. 6,287,598 to Ayer et al., U.S. Pat. No. 6,287,295 to Chen et al., U.S. Pat. No. 6,270,787 to Ayer et al., U.S. Pat. No. 6,261,584 to Peery et al., U.S. Pat. No. 6,245,357 to Edgren et al., U.S. Pat. No. 6,224,907 to Davar et al., U.S. Pat. No. 6,217,905 to Edgren et al., U.S. Pat. No. 6,210,712 to Edgren et al., U.S. Pat. No. 6,183,466 to Wong et al., U.S. Pat. No. 6,180,129 to Magruder et al., U.S. Pat. No. 6,146,662 to Jao et al., U.S. Pat. No. 6,132,420 to Dionne et al., U.S. Pat. No. 6,120,803 to Wong et al., U.S. Pat. No. 6,077,538 to Merrill et al., U.S. Pat. No. 6,020,000 to Wong et al., U.S. Pat. No. 6,007,837 to Enscore et al., U.S. Pat. No. 5,980,943 to Ayer et al., U.S. Pat. No. 5,938,654 to Wong et al., U.S. Pat. No. 5,914,131 to Merrill et al., U.S. Pat. No. 5,912,268 to Guittard et al., U.S. Pat. No. 5,869,096 to Barclay et al., U.S. Pat. No. 5,800,422 to Dong et al., U.S. Pat. No. 5,667,804 to Wong et al., U.S. Pat. No. 5,650,170 to Wright et al., U.S. Pat. No. 5,531,796 to Wong et al., U.S. Pat. No. 5,512,293 to Landrau et al., U.S. Pat. No. 5,498,255 to Wong, U.S. Pat. No. 5,460,826 to Merrill et al., U.S. Pat. No. 5,456,679 to Balaban et al., U.S. Pat. No. 5,443,459 to Wong et al., U.S. Pat. No. 5,324,280 to Wong et al., U.S. Pat. No. 5,320,616 to Magruder et al., U.S. Pat. No. 5,312,390 to Wong, U.S. Pat. No. 5,246,710 to Ayer et al., and U.S. Pat. No. 5,059,423 to Magruder et al., all of which are incorporated herein by this reference.

In the formulation of U.S. Pat. No. 6,764,697 to Jao et al., the formulation can comprise: (1) a core including the active beta-adrenergic inverse agonist, and (2) a layer substantially surrounding the core, the layer comprising hydroxyethylcellulose having a molecular weight of 8500 to 4,000,000; the layer delaying the release of the active beta-adrenergic inverse agonist from the core until a specified interval after administration dependent upon the weight of the layer.

In the formulation of U.S. Pat. No. 6,548,083 to Wong et al., the formulation can comprise: (1) a reservoir comprising an active beta-adrenergic inverse agonist, the reservoir being adapted to deliver the active beta-adrenergic inverse agonist over a prolonged period to a stomach of a subject; and (2) a layer of polymer matrix provided around the reservoir and including a swellable, water-soluble polymer and a water insoluble hydroattractant, the layer of polymer matrix being configured to promote gastric retention of the dosage form.

In the case of the oral chewable controlled-release formulation, controlled-release of the active beta-adrenergic inverse agonist is achieved despite the oral crushing of the tablets or capsules which would normally disrupt controlled delivery of a drug. An oral chewable controlled-release formulation of the active beta-adrenergic inverse agonist for the treatment of pulmonary airway diseases would be beneficial since nearly one third of all subjects with asthma in the United States are pediatric subjects. Pediatric subjects generally have a more difficult time swallowing pills whole. If they inadvertently chewed on an oral controlled-release formulation that required physical intactness to control drug release, such as is the case for an oral osmotic pump formulation, then they could potentially release of all of the contents of the formulation at one time. Since excessive levels, supra-therapeutic, of active beta-adrenergic inverse agonist may result in excessive occupancy of the beta adrenergic receptors of the pulmonary airways, this may result in bronchoconstriction or bronchospasm. The occurrence of uncontrolled bronchoconstriction could be life threatening. By providing an oral chewable controlled-release formulation, this allows pediatric and other patients an effective controlled-release formulation despite their difficulty swallowing tablets or capsules whole. Additionally, a chewable form may also taste good and be fun to take thereby improving patient compliance. Several approaches incorporating beta-adrenergic inverse agonists can be used to achieve the previously described drug pharmacokinetic profile over time such as those approaches disclosed in U.S. Pat. No. 6,248,363 to Patel et al. and in U.S. Pat. No. 5,853,762 to Myers et al., both of which are incorporated herein by this reference.

The formulations disclosed in U.S. Pat. No. 6,248,363 to Patel et al., in general, are in the form of a solid carrier comprising a substrate and an encapsulation coat on the substrate, wherein the encapsulation coat comprises an admixture of a therapeutically effective amount of active beta-adrenergic inverse agonist, an effective solubilizing amount of at least one hydrophilic surfactant, and a lipophilic additive selected from the group consisting of lipophilic surfactants, triglycerides, and combinations thereof, wherein the effective solubilizing amount of the at least one hydrophilic surfactant is an amount effective to partially or fully solubilize the active beta-adrenergic inverse agonist in the encapsulation coat. Alternatively, the formulation can be in the form of a solid carrier comprising an admixture of active beta-adrenergic inverse agonist, an effective solubilizing amount of at least one hydrophilic surfactant, and a lipophilic additive selected from the group consisting of lipophilic surfactants, triglycerides, and combinations thereof, wherein the effective solubilizing amount of the at least one hydrophilic surfactant is an amount effective to partially or fully solubilize the active beta-adrenergic inverse agonist in the solid carrier. In one alternative, the at least one hydrophilic surfactant comprises a non-ionic hydrophilic surfactant having an HLB value of at least about 10. Typically, the non-ionic hydrophilic surfactant is selected from the group consisting of alkylglucosides; alkylmaltosides; alkylthioglucosides; lauryl macrogolglycerides; polyoxyethylene alkyl ethers; polyoxyethylene alkylphenols; polyethylene glycol fatty acids esters; polyethylene glycol glycerol fatty acid esters; polyoxyethylene sorbitan fatty acid esters; polyoxyethylene-polyoxypropylene block copolymers; polyglycerol fatty acid esters; polyoxyethylene glycerides; polyoxyethylene sterols, derivatives, and analogues thereof; polyoxyethylene vegetable oils; polyoxyethylene hydrogenated vegetable oils; reaction mixtures of polyols and at least one member of the group consisting of fatty acids, glycerides, vegetable oils, hydrogenated vegetable oils, and sterols; tocopherol polyethylene glycol succinates; sugar esters; sugar ethers; sucroglycerides; and mixtures thereof. Alternatively, the at least one hydrophilic surfactant comprises an ionic surfactant, such as an ionic surfactant selected from the group consisting of alkyl ammonium salts; bile acids and salts, analogues, and derivatives thereof; fatty acid derivatives of amino acids, carnitines, oligopeptides, and polypeptides; glyceride derivatives of amino acids, oligopeptides, and polypeptides; acyl lactylates; mono- or di-acetylated tartaric acid esters of mono- or di-glycerides; succinylated monoglycerides; citric acid esters of mono- or di-diglycerides; alginate salts; propylene glycol alginate; lecithins and hydrogenated lecithins; lysolecithin and hydrogenated lysolecithins; lysophospholipids and derivatives thereof; phospholipids and derivatives thereof; salts of alkylsulfates; salts of fatty acids; sodium docusate; and mixtures thereof.

The substrate can be a powder or a multiparticulate.

The formulation disclosed in U.S. Pat. No. 5,853,762 to Myers et al. is, in general, a comestible unit including: (A) a controlled-release system, which disperses quickly in the mouth, prepared by a process comprising: (1) initiating crystallization of a shearform matrix; (2) before or after initiating crystallization combining a controlled-release system with the shearform matrix to form flowable, compactible micro-particulates; and (3) compacting the combination resulting from step (2) which includes at least partially crystallized shearform matrix, to form the unit; and (B) an active beta-adrenergic inverse agonist. The combining can comprise subjecting the controlled-release system and the matrix to treatment with a crystallization/binding promoter. The promoter can comprise an ingredient selected from the group consisting of an alcohol, polyvinylpyrrolidone, and a mixture thereof.

In the case of the dermal controlled-release patch formulation, controlled release of the beta-adrenergic inverse agonist can be achieved by using methods and compositions disclosed in U.S. Pat. No. 6,638,528 to Kanios, U.S. Pat. No. 6,024,974 to Li, U.S. Pat. No. 5,958,446 to Miranda et al., U.S. Pat. No. 5,719,197 to Kanios et al., U.S. Pat. No. 5,686,099 to Sablotsky et al., U.S. Pat. No. 5,656,285 to Sablotsky et al., U.S. Pat. No. 4,994,267 to Sablotsky et al., and U.S. Pat. No. 4,814,168 to Sablotsky et al., all of which are incorporated herein by this reference. In one approach, as described in U.S. Pat. No. 6,638,528 to Kanios, the composition comprises a pharmaceutically acceptable pressure-sensitive adhesive matrix consisting essentially of a blend of: (1) one or more adhesives selected from the group consisting of polyacrylates, polysiloxanes, polyisobutylene, polyisoprene, styrenes, styrene block copolymers and block amide copolymers in an amount from 20% to 75% by weight based on the dry weight of the total adhesive matrix composition; (2) an insoluble, non-adhesive ethyl cellulose polymer having a solution viscosity in the range of 3 cps to 40 cps, alone or in combination with an insoluble, non-adhesive cellulose ester in a total amount from 2.5% to 20% by weight based on the dry weight of the total adhesive matrix composition, (3) an active beta-adrenergic inverse agonist; (4) a hydrophilic crystallization inhibitor selected from the group consisting of soluble polyvinylpyrrolidone, soluble cellulose and cellulose derivatives, and polyethylene oxide in an amount from 5% to 15% by weight based on the dry weight of the total adhesive matrix composition; (5) a drug permeation enhancer in an amount up to 15% by weight based on the dry weight of the total adhesive matrix composition, and (6) a polyhydric alcohol solvent in an amount up to 20% by weight based on the dry weight of the total adhesive matrix composition; wherein the adhesive matrix is capable of delivering the active beta-adrenergic inverse agonist at a substantially zero-order kinetic rate for period of time in excess of 72 hours. In another-approach, the formulation comprises: (1) a therapeutically effective amount of an active beta-adrenergic inverse agonist; (2) a pharmaceutically acceptable carrier; and (3) a penetration enhancing amount of a functional derivative of a fatty acid, wherein the functional derivative of the fatty acid is selected from the group consisting of amides, alcohols, and polyols. In another approach, the formulation comprises a blend of: (1) a polyacrylate and a second polymer selected from the group consisting of a polysiloxane and a hydrocarbon polymer; and (2) a therapeutically effective amount of an active beta-adrenergic inverse agonist, wherein the composition is a pressure-sensitive adhesive and the polyacrylate and the second polymer modulates the permeation rate of the active beta-adrenergic inverse agonist through the dermis. In still another approach, the formulation comprises an active beta-adrenergic inverse agonist, a multipolymer containing vinyl acetate and ethylene monomers, a natural or synthetic rubber, and a tackifying agent, in which the ratio by weight of the multi-polymer to the rubber is about 1:1 to about 10:1.

Additional transdermal devices and formulations suitable for the delivery of active beta-adrenergic inverse agonist are disclosed in U.S. Pat. No. 6,893,655 to Flanigan et al., U.S. Pat. No. 6,312,715 to Cantor et al., U.S. Pat. No. 6,132,760 to Hedenstrom et al., U.S. Pat. No. 6,086,911 to Godbey, and U.S. Pat. No. 5,614,210 to Braun, all of which are incorporated herein by this reference.

Still additional transdermal therapeutic systems are described, for example, in Y. W. Chien, “Transdermal Therapeutic Systems” in Controlled Drug Delivery: Fundamentals and Applications (J. R. Robinson & V. H. L. Lee, eds, 2d ed., Marcel Dekker, New York, 1987), ch. 12, pp. 523-552, incorporated herein by this reference.

Another approach is to incorporate the drug into a slow-diffusing matrix of high viscosity as recited in U.S. Pat. No. 6,413,536 to Gibson et al. and in PCT Patent Publication No. WO 04/054542 (Yum et al.), both of which are incorporated herein by reference.

In the approach described in U.S. Pat. No 6,413,536 to Gibson et al, the beta-adrenergic inverse agonist is typically incorporated in a liquid composition that includes a non-water-soluble, high viscosity, liquid carrier material comprising a nonpolymeric ester or mixed ester of one or more carboxylic acids, having a viscosity of at least 5,000 centipoises at 37° C. and that does not crystallize neat under ambient or physiological conditions. The term “non-water soluble” refers to a material that is soluble in water to a degree of less than one percent by weight under ambient conditions. The term “non-polymeric” refers to esters or mixed esters having essentially no repeating units in the acid moiety of the ester, as well as esters or mixed esters having acid moieties wherein functional units in the acid moiety are repeated a small number of times (i.e., oligomers). Generally, materials having more than five identical and adjacent repeating units or mers in the acid moiety of the ester are excluded by the term “nonpolymeric” as used herein, but materials containing dimers, trimers, tetramers, or pentamers are included within the scope of this term. When the ester is formed from hydroxy-containing carboxylic acid moieties that can further esterify, such as lactic acid or glycolic acid, the number of repeat units is calculated based upon the number of lactide or glycolide moieties, rather than upon the number of lactic acid or glycolic acid moieties, where a lactide repeat unit contains two lactic acid moieties esterified by their respective hydroxy and carboxy moieties, and where a glycolide repeat unit contains two glycolic acid moieties esterified by their respective hydroxy and carboxy moieties. Esters having 1 to about 20 etherified polyols in the alcohol moiety thereof, or 1 to about 10 glycerol moieties in the alcohol moiety thereof, are considered nonpolymeric as that term is used herein. The high viscosity liquid carrier material can decrease in viscosity, sometimes significantly, when mixed with a solvent to form a low viscosity liquid carrier material, which can be in turn mixed with the beta-adrenergic inverse agonist.

The particular high viscosity liquid carrier material (“HVLCM”) used in the invention can be one or more of a variety of materials. Suitable materials include nonpolymeric esters or mixed esters of one or more carboxylic acids. In a particular embodiment, the ester is formed from carboxylic acids that are esterified with a polyol having from about 2 to about 20 hydroxy moieties, and which may include 1 to about 20 etherified polyols. Particularly suitable carboxylic acids for forming the acid moiety of the ester of the HVLCM include carboxylic acids having one or more hydroxy groups, e.g., those obtained by ring opening alcoholysis of lactones, or cyclic carbonates or by the alcoholysis of carboxylic acid anhydrides. Amino acids are also suitable for forming esters with the polyol. In a particular embodiment, the ester or mixed ester contains an alcohol moiety having one or more terminal hydroxy moieties that have been esterified with one or more carboxylic acids obtained by alcoholysis of a carboxylic acid anhydride, such as a cyclic anhydride.

Nonlimiting examples of suitable carboxylic acids that can be esterified to form the HVLCM that can be used for the preparation of controlled-release preparations of beta-adrenergic inverse agonists according to the present invention include glycolic acid, lactic acid, ε-hydroxycaproic acid, serine, and any corresponding lactones or lactams, trimethylene carbonate, and dioxanone. The hydroxy-containing acids may themselves be further esterified through the reaction of their hydroxy moieties with additional carboxylic acid, which may be the same as or different from other carboxylic acid moieties in the material. Suitable lactones include, but are not limited to, glycolide, lactide, ε-caprolactone, butyrolactone, and valerolactone. Suitable carbonates include but are not limited to trimethylene carbonate and propylene carbonate.

The alcohol moiety of the ester or mixed ester may be derived from a polyhydroxy alcohol having from about 2 to about 20 hydroxy groups, and as indicated above, may be formed by etherifying 1 to 20 polyol molecules. Suitable alcohol moieties include those derived by removing one or more hydrogen atoms from: monofunctional C₁-C₂₀ alcohols, difunctional C₁-C₂₀ alcohols, trifunctional alcohols, hydroxy-containing carboxylic acids, hydroxy-containing amino acids, phosphate-containing alcohols, tetratunctional alcohols, sugar alcohols, monosaccharides, and disaccharides, sugar acids, and polyether polyols. More specifically, the alcohol moieties may include one or more of: dodecanol, hexanediol, more particularly, 1,6-hexanediol, glycerol, glycolic acid, lactic acid, hydroxybutyric acid, hydroxyvaleric acid, hydroxycaproic acid, serine, ATP, pentaerythritol, mannitol, sorbitol, glucose, fructose, sucrose, glucuronic acid, polyglycerol ethers containing from 1 to about 10 glycerol units, and polyethylene glycols containing 1 to about 20 ethylene glycol units. In particular embodiments of the invention, at least one of the carboxylic acid moieties of the esters or mixed esters of the invention comprise at least one oxy moiety. In an even more particular embodiment, each of the carboxylic acid moieties comprise at least one oxy moiety. In another particular embodiment, at least one of the carboxylic acid moieties of the esters or mixed esters of the invention contains 2 to 4 carbon atoms. In an even more particular embodiment, each of the carboxylic acid moieties of the esters or mixed esters of the invention contains 2 to 4 carbon atoms. In another more particular embodiment of the invention, at least one of the carboxylic acid moieties of the ester or mixed ester of the invention has 2 to 4 carbon atoms and contains at least one oxy moiety. In another more particular embodiment of the invention, each of the carboxylic acid moieties of the ester or mixed ester of the invention has 2 to 4 carbon atoms and contains at least one oxy moiety.

In general, the HVLCM esters of the invention can be made by reacting one or more alcohols, in particular one or more polyols, which will form the alcohol moiety of the resulting esters with one or more carboxylic acids, lactones, lactams, carbonates, or anhydrides of the carboxylic acids which will form the acid moieties of the resulting esters. The esterification reaction can be conducted simply by heating, although in some instances addition of a strong acid or strong base esterification catalyst may be used. Alternatively, an esterification catalyst such as stannous 2-ethylhexanoate can be used. The heated reaction mixture, with or without catalyst, is heated with stirring, then dried, e.g., under vacuum, to remove any unreacted starting materials, to produce a liquid product. Sucrose acetate isobutyrates can be made by following the procedures described in U.S. Pat. No. 2,931,802.

As described above, in one embodiment of the invention, the HVLCM can be mixed with a viscosity lowering solvent to form a lower viscosity liquid carrier material (LVLCM), which can then be mixed with the biologically active substance to be delivered, prior to administration. These solvents can be water soluble, non-water soluble, or water miscible, and can include acetone, benzyl alcohol, benzyl benzoate, N-(betahydromethyl) lactamide, butylene glycol, caprolactam, caprolactone, corn oil, decylmethylsulfoxide, dimethyl ether, dimethyl sulfoxide, 1-dodecylazacycloheptan-2-one, ethanol, ethyl acetate, ethyl lactate, ethyl oleate, glycerol, glycofurol (tetraglycol), isopropyl myristate, methyl acetate, methyl ethyl ketone, N-methyl-2-pyrrolidone, MIGLYOLs (esters of caprylic and/or capric acids with glycerol or alkylene glycols, e.g., MIGLYOL 810 or 812 (caprylic/capric triglycerides), MIGLYOL 818 (caprylic/capric/linoleic triglyceride), MIGLYOL 829 (caprylic/capric/succinic triglyceride), MIGLYOL 840 (propylene glycol dicaprylate/caprate)), oleic acid, peanut oil, polyethylene glycol, propylene carbonate, 2-pyrrolidone, sesame oil, SOLKETAL ((±)-2,2-dimethyl-1,3-dioxolane-4-methanol), tetrahydrofuran, TRANSCUTOL (diethylene glycol monoethyl ether, carbitol), triacetin, triethyl citrate, and combinations thereof.

When the composition is used as a LVLCM in conjunction with administration of a biologically active substance, it should contain a solvent that the HVLCM is soluble in. In certain instances, the substance to be delivered is also soluble in the solvent. The solvent should be non-toxic and otherwise biocompatible. Solvents that are toxic should not be used for pharmaceutical or agricultural purposes. The solvents used to inject the composition into animals should not cause significant tissue irritation or necrosis at the site of implantation, unless irritation or necrosis is the desired effect.

In one embodiment, the solvent should be at least water soluble, so that it will diffuse quickly into bodily fluids or other aqueous environment, causing the composition to coagulate or solidify. In another embodiment, the solvent is not completely miscible with water or bodily fluids so that diffusion of the solvent from the composition, and the corresponding increase in viscosity of the composition, are slowed.

When esters of 1,6-hexanediol or glycerol are used as the HVLCM, some possible solvents are ethanol, N-methylpyrrolidone, propylene carbonate, and PEG 400.

The solvent is typically added to the compositions in an amount in the range from about 1 percent to about 95 percent by weight, more particularly from about 5 to about 90 weight percent, relative to the total weight of the composition. Even more particularly, the solvent is present in the composition in an amount in the range from about 10 percent to about 55 percent by weight. Other particular ranges include from about 10 percent to 50 percent by weight, and from about 10 to about 30 percent by weight.

A further embodiment involves the use of solvents that are not solvents for the HVLCM such that when combined with the HVLCM singularly or in combination with a solvent for the HVLCM, the resulting composition forms an emulsion. Such emulsions may contain the HVLCM in the dispersed phase such as in the case of SAIB/MIGLYOL mixtures that are emulsified in water or glycerol, or they may contain the HVLCM as a component of the continuous phase such as in the case of an aqueous solution that is emulsified in the HVLCM or a solution of the HVLCM in a water immiscible solvent.

Moreover, the formulations containing biologically active substances and an HVLCM or LVLCM may be further formulated with polymeric excipients to provide a drug delivery matrix with modified properties, for example a faster or slower degradation rate. The resulting composition may be formed into microspheres, or into a macroscopic implant, or other geometries and sizes according to techniques known in the art. Alternatively, a pre-formed microsphere or implant with a biologically active substances incorporated therein can be combined with the HVLCM or LVLCM, for example as an injection vehicle. Here the HVLCM or LVLCM will form a secondary barrier to provide enhanced drug delivery. The HVLCM or LVLCM phase may or may not contain other biologically active substances, according to the specific biological requirement. These other biologically active substances may be any of those described above, provided that the biologically active substance must be suitable for incorporation into microspheres or implants according to techniques known in the art.

A variety of additives can optionally be added to the HVLCM or LVLCM to modify the properties of the material as desired, and in particular to modify the release properties of the composition with respect to the beta-adrenergic inverse agonist. The additives can be present in any amount which is sufficient to impart the desired properties to the composition. The amount of additive used will in general be a function of the nature of the additive and the effect to be achieved, and can be easily determined by one of ordinary skill in the art. Suitable additives are described in U.S. Pat. No. 5,747,058, the entire contents of which are hereby incorporated by reference. More particularly, suitable additives include water, biodegradable polymers, non-biodegradable polymers, natural oils, synthetic oils, carbohydrates or carbohydrate derivatives, inorganic salts, BSA (bovine serum albumin), surfactants, organic compounds, such as sugars, and organic salts, such as sodium citrate. Some of these classes of additives are described in more detail below. In general, the less water soluble, i.e., the more lipophilic, the additive, the more it will decrease the rate of release of the substrate, compared to the same composition without the additive. In addition, it may be desirable to include additives that increase properties such as the strength or the porosity of the composition.

The addition of additives can also be used to lengthen the delivery time for the active ingredient, making the composition suitable for treatment of disorders or conditions responsive to longer term administration. Suitable additives in this regard include those disclosed in U.S. Pat. No. 5,747,058. In particular, suitable additives for this purpose include polymeric additives, such as cellulosic polymers and biodegradable polymers. Suitable cellulosic polymers include cellulose acetates, cellulose ethers, and cellulose acetate butyrates. Suitable biodegradable polymers include polylactones, polyanhydrides, and polyorthoesters, in particular, polylactic acid, polyglycolic acid, polycaprolactone, and copolymers thereof.

When present, the additive is typically present in the compositions in an amount in the range from about 0.01 percent to about 20 percent by weight, more particularly from about 0.1 percent to about 20 percent by weight, relative to the total weight of the composition, and more typically, is present in the composition in an amount in the range from about 1, 2, or 5 percent to about 10 percent by weight. Certain additives, such as buffers, are only present in small amounts in the composition.

The following categories are nonlimiting examples of classes of additives that can be employed in this embodiment of compositions according to the present invention. Given the disclosure herein and the objects to be achieved, one of skill in the art will easily know how to select other additives to achieve a desired purpose. All of these embodiments are considered to fall within the disclosed invention: (1) Biodegradable Polymers. One category of additives is biodegradable polymers and oligomers. The polymers can be used to alter the release profile of the substance to be delivered, to add integrity to the composition, or to otherwise modify the properties of the composition. Non-limiting examples of suitable biodegradable polymers and oligomers include: poly(lactide), poly(lactide-co-glycolide), poly(glycolide), poly(caprolactone), polyamides, polyanhydrides, polyamino acids, polyorthoesters, polycyanoacrylates, poly(phosphazines), poly(phosphoesters), polyesteramides, polydioxanones, polyacetals, polyketals, polycarbonates, polyorthocarbonates, degradable polyethylenes, polyhydroxybutyrates, polyhydroxyvalerates, polyalkylene oxalates, polyalkylene succinates, poly(malic acid), chitin, chitosan, and copolymers, terpolymers, oxidized cellulose, or combinations or mixtures of the above materials. Examples of poly(α-hydroxy acid)s include poly(glycolic acid), poly(DL-lactic acid) and poly(L-lactic acid), and their copolymers. Examples of polylactones include poly(ε-caprolactone), poly(δ-valerolactone) and poly(γ-butyrolactone). (2) Non-biodegradable Polymers. Another class of additives for use with the present compositions is non-biodegradable polymers. Non-limiting examples of nonerodible polymers which can be used as additives include: polyacrylates, ethylene-vinyl acetate polymers, cellulose and cellulose derivatives, acyl substituted cellulose acetates and derivatives thereof, non-erodible polyethylenes, polystyrenes, polyvinyl chloride, polyvinyl fluoride, polyvinyl (imidazole), chlorosulfonated polyolefins, polyethylene oxide, and polyethylene. Preferred non-biodegradable polymers include polyvinyl pyrrolidone, ethylene vinylacetate, polyethylene glycol, cellulose acetate butyrate (“CAB”) and cellulose acetate propionate (“CAP”). (3) Oils and Fats A further class of additives which can be used in the present compositions are natural and synthetic oils and fats. Oils derived from animals or from plant seeds of nuts typically include glycerides of the fatty acids, chiefly oleic, palmitic, stearic, and linoleic. As a rule the more hydrogen the molecule contains the thicker the oil becomes. Non-limiting examples of suitable natural and synthetic oils include vegetable oil, peanut oil, medium chain triglycerides, soybean oil, almond oil, olive oil, sesame oil, peanut oil, fennel oil, camellia oil, corn oil, castor oil, cotton seed oil, and soybean oil, either crude or refined, and medium chain fatty acid triglycerides. Fats are typically glyceryl esters of higher fatty acids such as stearic and palmitic. Such esters and their mixtures are solids at room temperatures and exhibit crystalline structure. Lard and tallow are examples. In general oils and fats increase the hydrophobicity of the HVLCM, slowing degradation and water uptake. (4) Carbohydrates and Carbohydrate Derivatives. Another class of additives which can be used in the present compositions is carbohydrates and carbohydrate derivatives. Non-limiting examples of these compounds include monosaccharides (simple sugars such as fructose and its isomer glucose (dextrose); disaccharides such as sucrose, maltose, cellobiose, and lactose; and polysaccharides.

Additionally, the composition can further include a network former and, optionally, a rheology modifier as disclosed in PCT Patent Publication No. WO 2004/054542 by Yum et al., published Jul. 1, 2004, incorporated in its entirety by this reference.

Suitable network formers include cellulose acetate butyrate, carbohydrate polymers, organic acids of carbohydrate polymers and other polymers, hydrogels, particles such as silicon dioxide, ion exchange resins, and/or fiberglass, that are capable of associating, aligning, or congealing to form three-dimensional networks in an aqueous environment. Other examples include cellulose acetate phthalate, ethylcellulose, Pluronic, Eudragit, Carbomer, hydroxypropylmethylcellulose, cellulose acetates, cellulose triacetate, PMMA, and CAB 500-5.

Suitable rheology modifiers include caprylic/capric triglycerides (e.g. Migliol 810), isopropyl myristate, ethyl oleate, triethyl citrate, dimethyl phthalate, and benzyl benzoate.

In another alternative, the formulation can be covered by an enteric coating such as an enteric polymer to delay any release of the drug contained in the composition until the formulation reaches the upper intestinal tract, where the coating is dissolved and ingredients in the composition, including the beta-adrenergic inverse agonist, are available for release and absorption into the bloodstream. Suitable enteric coatings are well known in the art and include, but are not limited to, cellulose acetate phthalate, cellulose acetate trimellitate, hydroxy propyl methyl cellulose acetate phthalate, hydroxy propyl methyl cellulose acetate succinate, carboxy methyl ethyl cellulose, polyvinyl acetate phthalate, copolymer of vinyl acetate and crotonic acid and poly(methacrylic acid, ethacrylate), and Eudragit® S12.5, Eudragit® S 100, Eudragit® FS 30D (all from Rohm), Sureteric® (from Colorcom), Aquateric® (from FMC) or HPMCP (from Shin-Etsu). Other enteric coatings include hydroxypropyl methylcellulose acetate, polyvinyl alcohol phthalate, and a copolymer of styrene and maleic acid. Still other enteric coatings include fatty acid mixtures. Generally, enteric coatings rapidly disintegrate or dissolve at pH values of 5 or above. Generally, formulations coated by enteric coatings consist of porous particles whose pores contain an active ingredient and a polymer acting as a blocking agent that degrades and releases the active ingredient upon exposure to either low or high pH or to changes in ionic strength. The most effective enteric materials include polyacids having a pKₐ of from about 3 to 5. The amount of coating applied is adapted so as to obtain a predetermined dissolution characteristic of the composition, which can be adjusted to obtain appropriate pharmacokinetic characteristics. Typically, the use of an enteric coating delays the onset of release of the active beta-adrenergic agonist by 2 hours or slightly longer. However, the amount of coating applied should also be adapted so that there will be no rupturing problems. The coating may be admixed with various excipients such as plasticizers, anti-adhesives such as, e.g., colloidal silicon dioxide, inert fillers, lipophilic agents such as, e.g, stearic acid, capric acid or hydrogenated castor oil, colon targeting excipients such as, e.g. amylose, ethylcellulose, Eudragit S 12.5, or other excipients. Enteric coatings are described in, for example, U.S. Pat. No. 7,070,803 to Skinhoj et al., U.S. Pat. No. 7,063,862 to Lin et al., U.S. Pat. No. 7,060,295 to Richardson et al., U.S. Pat. No. 7,056,942 to Hildesheim et al., and U.S. Pat. No. 5,316,774 to Eury et al., all incorporated herein by this reference.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-α-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, especially as described below under combination therapy. For administration of any of the compounds used in the present invention, preparations should meet sterility, pyrogenicity, general safety, and purity standards as required by the FDA Office of Biologics Standards or by other regulatory organizations regulating drugs.

Other controlled-release devices and methods, including implantable devices, are known in the art.

The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by this reference.

One of ordinary skill in the art can readily prepare formulations for controlled release or sustained release comprising an active beta-adrenergic inverse agonist by modifying the formulations described above, such as according to principles disclosed in V. H. K. Li et al, “Influence of Drug Properties and Routes of Drug Administration on the Design of Sustained and Controlled Release Systems” in Controlled Drua Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the active beta-adrenergic inverse agonist, such as aqueous solubility, partition coefficient, molecular size, stability of the inverse agonist, and binding of the inverse agonist to proteins and other biological macromolecules. This process of preparation also takes into account biological factors, such as absorption, distribution, metabolism, duration of action, the possible existence of side effects, and margin of safety, for the inverse agonist. Accordingly, one of ordinary skill in the art could modify the formulations in order to incorporate an active beta-adrenergic inverse agonist into a formulation having the desirable properties described above for a particular application.

Typically, the average Tₘₐₓ is greater than about 4 hours. Preferably, the average Tₘₐₓ is greater than about 8 hours. In one alternative, the average Tₘₐₓ is preferably from about 8 hours to about 12 hours. In yet another alternative, the average Tₘₐₓ is preferably from about 12 hours to about 16 hours. In another alternative, the average Tₘₐₓ is preferably from about 16 hours to about 24 hours. In still another alternative, the average Tₘₐₓ is preferably about 24 hours.

Typically, the average half-life of the active beta-adrenergic inverse agonist in the blood is >16 hours when a formulation according to the present invention is administered with once-daily dosing. In one alternative, the average half-life is preferably from about 18 hours to about 22 hours. In yet another alternative, the average half-life is preferably from about 22 to 28 hours. In still another alternative, the average half-life is preferably from about 28 to 38 hours. In the case of dermal controlled release formulations, the average half-life may range from 24 hours up to 10 days.

When the first dose of non-sustained release nadolol is administered at a dosage of 80 mg for a period of 24 hours dosing interval, the average Cₘₐₓ is 132 ng/mL in the blood, while the average Cₘᵢₙ is 30 ng/mL in the blood (ANDA 074229 Zenith Goldline Pharmaceuticals, Inc.). Typically, formulations according to the present invention reduce this ratio of Cₘₐₓ/Cₘᵢₙ with the first dose from about 4.4 to less than 4.0 and more preferably to about 2.0 or less, particularly when the active beta-adrenergic inverse agonist is nadolol. Preferably, they reduce this ratio of Cₘₐₓ/Cₘᵢₙ to about 1.5 or less. More preferably, they reduce this ratio of Cₘₐₓ/Cₘᵢₙ to about 1.33 or less.

One embodiment of the invention is a formulation of a controlled-release formulation, as described above, containing a beta-adrenergic inverse agonist or a combination of a beta-adrenergic inverse agonist with a second drug for the purpose of treatment of pulmonary airway disease in a subject suffering from pulmonary airway disease as recited in PCT Patent Application Serial No. PCT/US2004/033157 to Bond, filed Oct. 8, 2004, incorporated herein in its entirety by this reference. Additionally, the subject may have two or more chronic co-morbid diseases such as a pulmonary airway disease with any of the following, hypertension, angina, congestive heart failure, cardiac arrhythmia, migraines, anxiety, tremor, rosacea, osteoporosis, or other cardiovascular diseases.

Typically, the beta-adrenergic inverse agonist has therapeutic activity against a pulmonary airway disease. The beta-adrenergic inverse agonist included in the controlled-release formulation as described above can, in a significant alternative, also have therapeutic activity against a co-morbid condition selected from the group consisting of hypertension, angina, congestive heart failure, cardiac arrhythmia, migraines, anxiety, tremor, rosacea, osteoporosis, and other cardiovascular diseases when administered to a subject that has a pulmonary airway disease or congestive heart failure together with one or more of these co-morbid conditions. This treats two diseases or conditions with one drug.

Preferably, the beta-adrenergic inverse agonist is selected from the group consisting of nadolol, bupranolol, butoxamine, carazolol, carvedilol, ICI-118,551, levobunolol, metoprolol, propranolol, sotalol, and timolol, and the salts, solvates, analogues, congeners, mimetics, bioisosteres, stereoisomers, hydrolysis products, metabolites, precursors, and prodrugs thereof. Particularly preferred beta-adrenergic inverse agonists are nadolol, sotalol, metoprolol, timolol, and ICI 118,551. Additionally preferred beta-adrenergic inverse agonists are the analogues of nadolol of formula (I) wherein R₁ is hydrogen or lower alkyl, R₂ is hydrogen or lower alkyl, and m and n are 1 to 3, with the proviso that where R₁ and R₂ are both hydrogen and m is 1, n is other than 1. As used herein, the term “lower alky” is defined as a straight or branched hydrocarbyl residue of 1-6 carbon atoms.

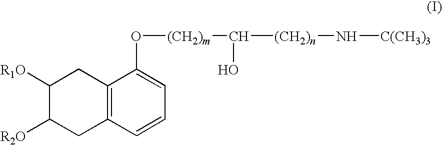

Still other additionally preferred beta-adrenergic inverse agonists are analogues of carvedilol of formula (II) wherein R₁ is hydrogen or lower alkyl, R₂ is hydrogen or lower alkyl, and R₃ is hydrogen or lower alkyl, with the proviso that all of R₁, R₂, and R₃ are not all hydrogen.

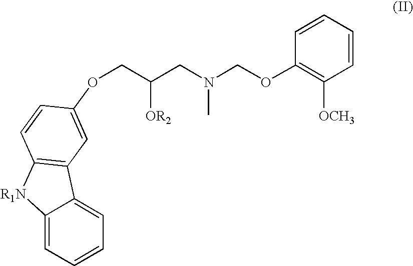

Also expected to be within the scope of the invention are analogues of timolol of formula (III) wherein R₁ is hydrogen or lower alkyl and R₂ is hydrogen or lower alkyl, with the proviso that both R₁ and R₂ are not hydrogen.

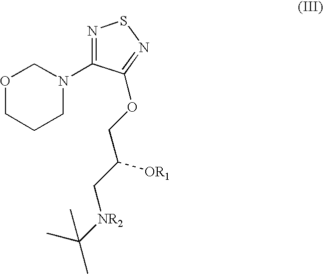

Further expected to be within the scope of the invention are analogues of metoprolol of formula (IV) wherein R₁ is hydrogen or lower alkyl and R₂ is hydrogen or lower alkyl, with the proviso that both R₁ and R₂ are not hydrogen.

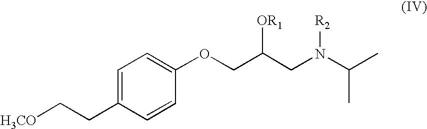

Also further expected to be within the scope of the invention are analogues of ICI-118,551 of formula (V) wherein R₁ is lower alkyl, R₂ is hydrogen or lower alkyl, R₉ is hydrogen or lower alkyl, R₄ is hydrogen or lower alkyl, R₅ is lower alkyl, and R₆ is lower alkyl, with the proviso that all of R₁, R₃, R₅, and R₆ are not methyl and all of R₂ and R₄ are not hydrogen.

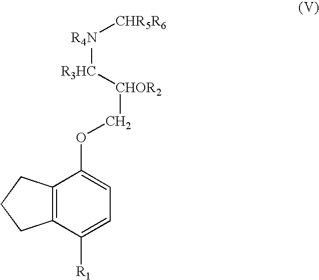

In the case of salts, it is well known that organic compounds, including compounds having activities suitable for methods according to the present invention, have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound or analogue includes such salt forms as occur at physiological pH or at the pH of a pharmaceutical composition unless specifically excluded.

Similarly, prodrug esters can be formed by reaction of either a carboxyl or a hydroxyl group on compounds or analogues suitable for methods according to the present invention with either an acid or an alcohol to form an ester. Typically, the acid or alcohol includes a lower alkyl group such as methyl, ethyl, propyl, isopropyl, butyl, isobutyl, and tertiary butyl. These groups can be substituted with substituents such as hydroxy, or other substituents. Such prodrugs are well known in the art and need not be described further here. The prodrug is converted into the active compound by hydrolysis of the ester linkage, typically by intracellular enzymes. Other suitable groups that can be used to form prodrug esters are well known in the art. For example prodrugs can include amides prepared by reaction of the parent acid compound with a suitable amine. In some cases it is desirable to prepare double ester type prodrugs such as (acyloxy) alkyl esters or ((alkoxycarbonyl)oxy)alkyl esters. Suitable esters as prodrugs include, but are not necessarily limited to, methyl, ethyl, propyl, isopropyl, n-butyl, isobutyl, tert-butyl, morpholinoethyl, and N,N-diethylglycolamido. Methyl ester prodrugs may be prepared by reaction of the acid form of a compound having a suitable carboxylic acid group in a medium such as methanol with an acid or base esterification catalyst (e.g., NaOH, H₂SO₄). Ethyl ester prodrugs are prepared in similar fashion using ethanol in place of methanol. Morpholinylethyl ester prodrugs may be prepared by reaction of the sodium salt of a suitable compound (in a medium such as dimethylformamide) with 4-(2-chloroethyl)morphine hydrochloride (available from Aldrich Chemical Co., Milwaukee, Wis. USA.

Pharmaceutically acceptable salts include acid salts such as hydrochlorides, hydrobromides, hydroiodides, sulfates, phosphates, fumarates, maleates, acetates, citrates, lactates, tartrates, sulfamates, malonates, succinates, tartrates, methanesulfonates, ethanesulfonates, benzenesulfonates, p-toluenesulfonates, cyclohexylsulfamates, quinates, formates, cinnamates, picrates, and other suitable salts. Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid, as well as other salts well known in the art.

Pharmaceutically acceptable salts also include salts with bases such as alkali metal salts such as sodium or potassium, as well as pyridine salts, ammonium salts, piperazine salts, diethylamine salts, nicotinamide salts, calcium salts, magnesium salts, zinc salts, lithium salts, methylamino salts, triethylamino salts, dimethylamino salts, and tris(hydroxymethyl)aminomethane salts.

As used herein, the term “active beta-adrenergic inverse agonist” is one or more chiral forms of the drug that binds to the beta adrenergic receptor as an inverse agonist whereas the inactive form chiral form does not have adrenergic receptor binding activity as an inverse agonist. The term “active beta-adrenergic inverse agonist” includes the active stereoisomer of chiral beta-adrenergic inverse agonists, and the salts and prodrugs of active beta-adrenergic inverse agonists unless specifically excluded. However, the term “active beta-adrenergic inverse agonist” also includes a racemic mixture of an active stereoisomer and its mirror image, unless specifically excluded. The term “active beta-adrenergic inverse agonist” also includes active metabolites of active beta-adrenergic inverse agonists that have substantially the same spectrum of pharmacological activity as the unmetabolized active beta-adrenergic inverse agonist unless specifically excluded.

Additionally, pharmaceutical compositions according to the present invention can further include conventional ingredients used in pharmaceutical compositions, such as lubricants in tablets or pills, coloring agents, or flavoring agents. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, or insofar as it affects the controlled-release properties of the composition, its use in a composition according to the present invention is contemplated.

The subject to be treated can be a human patient or a socially or economically important animal, including, but not limited to, a dog, a cat, a horse, a sheep, a cow, a goat, or a pig. Methods according to the present invention are not limited to the treatment of humans.

In this composition, the beta-adrenergic inverse agonist is formulated to be controlled release as to be optimal for the treatment of pulmonary airway diseases and minimizing acute side effects as compared to an un-controlled or immediate-release form of the drug into patients with pulmonary airway disease.

The beta-adrenergic inverse agonist can be suitable for the treatment of a pulmonary airway condition as described above. The beta-adrenergic inverse agonist included in the controlled-release formulation can, in a significant alternative, have therapeutic activity against a co-morbid condition selected from the group consisting of hypertension, angina, congestive heart failure, cardiac arrhythmia, migraines, anxiety, tremor, rosacea, osteoporosis, and other cardiovascular diseases when administered to a subject that has a pulmonary airway disease together with one or more of these co-morbid conditions, as described above. This treats both diseases or conditions simultaneously by administration of one drug.

The beta-adrenergic inverse agonist can be supplied in an oral dosage form comprising a formulation contained within a biodegradable capsule, wherein the formulation is as described above. The biodegradable capsule can be chosen to influence the pharmacokinetic properties of the composition. Typically, in this alternative, the capsule is made of a substance that degrades when exposed to conditions present in the gastrointestinal tract of a mammal, such as a human or a non-human mammal. In certain embodiments the capsule comprises gelatin or synthetic polymers such as hydroxyethylcellulose or hydroxypropylmethylcellulose. Gelcaps can be used; the gelcaps can be of the hard or soft variety. Gelatin capsules are suitable for delivering liquid formulations. In some embodiments, the dosage form can comprise at least one additional component selected from the group consisting of ethyl lactate, triacetin, propylene carbonate, glycofurol, triethyl oleate, isopropyl myristate, cellulose acetate butyrate, and derivatives of these compounds.

In some cases, a second drug for treatment of the pulmonary airway disease can be administered. The second drug for treatment of the pulmonary airway disease can be a beta₂-selective adrenergic agonist, a steroid, an anticholinergic, a methylxanthine compound, an anti-IgE antibody, a leukotriene modifier, or a phosphodiesterase IV inhibitor. This can be included in the controlled-release formulation or administered separately, as desired.

Typically, the controlled-release formulation is formulated for once-daily administration. However, a controlled-release formulation according to the present invention can be formulated for administration at different intervals, such as twice, three times, or four times daily, or for administration at longer intervals, such as two days, three days, four days, one week, or two weeks. One of ordinary skill in the art can modify the formulations described above to obtain a suitable formulation for the interval of administration intended.

The pulmonary airway disease to be treated can be selected from the group consisting of asthma, bronchiectasis, bronchitis, chronic obstructive pulmonary disease, Churg-Strauss syndrome, the pulmonary sequelae of cystic fibrosis, emphysema, allergic rhinitis, and pneumonia. Typically, the pulmonary airway disease is asthma.

In one embodiment, a controlled-release formulation according to the present invention is formulated to reduce serum blood levels of the active beta-adrenergic inverse agonist, such as nadolol, over a time period from about 2 a.m. to about 6 a.m. in order to treat nocturnal asthma. Nocturnal asthma has recently been recognized as a serious problem affecting a significant fraction of asthma sufferers (P. E. Silkoff & R. J. Martin, “Pathophysiology of Nocturnal Asthma,” Ann. Allergy Asthma Immunol. 81: 378-387 (1998)). A study of more than 7,000 asthma patients found that 74% of those surveyed awakened from sleep at least once weekly with symptoms requiring inhaled beta₂-agonist use; such symptoms included wheezing, chest tightness, or cough. Almost 40% awakened nightly due to asthma, and 64% reported awakening three times or more weekly. Nocturnal asthma is generally believed to be characteristic of a more serious disease process. It is believed that nocturnal asthma is related to the effect of the circadian rhythm on pulmonary function. Both normal and asthmatic subjects have maximum pulmonary function around 4 p.m. and minimum pulmonary function around 4 a.m., but the changes are much larger in asthmatic subjects and are associated with a change in non-specific airway reactivity. Circadian fluctuations in hormone levels may also play a role. Therefore, formulations and methods suitable for treatment of nocturnal asthma are highly desirable.

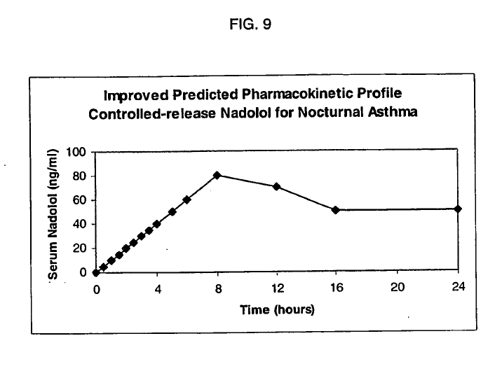

In particular, formulations of beta-adrenergic inverse agonists, such as nadolol, suitable for the treatment of nocturnal asthma typically have the pharmacokinetic profile shown in FIG. 9 in Example 6, below (a predicted example, not based on actual results). For this profile the average Tₘₐₓ is 8 hours and the concentration of the active beta-adrenergic inverse agonist, such as nadolol, in the blood ascends gradually up to this time point (8 hours after administration) and then gradually descends for the next 8 hours and then stays flat for the last 8 hours of a 24-hour cycle. This pharmacokinetic profile is based on morning dosing typically at 8 a.m. However, for patients that sleep at other times of the day, such as during the daytime for patients that work night shifts, their dosing regime would be correlated with their waking periods and not with the time of day. It is obvious to those skilled in the art that the absolute levels of the drug dose can easily be reduced or increased but still maintain the same shape of the curve over the 24 hour time period.

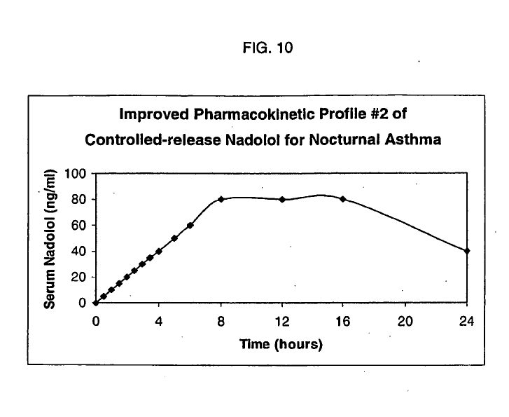

Another embodiment of the invention is a formulation that is also suitable for the treatment of nocturnal asthma symptoms, particularly for symptoms from 2 a.m. to 6 a.m. For such a formulation, formulations typically have the pharmacokinetic profile shown in FIG. 10 in Example 7, below (a predicted example, not based on actual results). For this profile the average Tₘₐₓ is about 8 hours and the concentration of the active beta-adrenergic agonist, such as nadolol, in the blood ascends gradually up to this time point (8 hours after administration) and then remains essentially constant in the bloodstream till 16 hours after dosing and then gradually descends for the last 8 hours of a 24-hour cycle. This is a predicted example, not based on actual results. This pharmacokinetic profile is for patients that have nocturnal asthma symptoms from 2 a.m. to 6 a.m. However, for patients that sleep at other times of the day, such as during the daytime for patients that work night shifts, their dosing regime would be correlated with their waking periods and not with the time of day. It is obvious to those skilled in the art that the absolute levels of the drug dose can easily be reduced or increased but still maintain the same shape of the curve over the 24 hour time period. Other variations in the pharmacokinetic profile can be devised by those of ordinary skill in the art and are within the scope of the present invention.

Another embodiment of the invention is a method of treatment of a pulmonary airway disease comprising administering a controlled-release formulation according to the present invention in a therapeutically effective quantity to a subject suffering from pulmonary airway disease. The subject can also suffer from a co-morbid disease or condition, in which case the beta-adrenergic inverse agonist in the controlled-release formulation can also be therapeutically effective against the co-morbid condition in the quantity that it is administered to the subject in the formulation, thus treating two diseases or conditions simultaneously with one drug. In another alternative, the controlled-release formulation can include a second drug for treatment of the pulmonary airway disease, as described above. The second drug can be, for example, a β₂-selective adrenergic agonist, a steroid, an anticholinergic, a methylxanthine compound, an anti-IgE antibody, a leukotriene modifier, or a phosphodiesterase IV inhibitor. β₂-selective adrenergic agonists suitable for use as the second drug include, but are not necessarily limited to, albuterol, bitolterol, clenbuterol, clorprenaline, dobutamine, fenoterol, formoterol, isoetharine, isoprenaline, levabuterol, mabuterol, metaproterenol, pirbuterol, ritodrine, salbutamol, salmeterol, and terbutaline, as well as the salts, solvates, analogues, congeners, mimetics, stereoisomers, bioisosteres, hydrolysis products, metabolites, precursors, and prodrugs thereof. Steroids suitable for the second drug include, but are not necessarily limited to, beclomethasone, budenoside, ciclesonide, flunisolide, fluticasone, methylprednisolone, prednisolone, prednisone, and triamcinolone, as well as the salts, solvates, analogues, congeners, mimetics, stereoisomers, bioisosteres, hydrolysis products, metabolites, precursors, and prodrugs thereof. Anticholinergics suitable for use as the second drug include, but are not necessarily limited to, muscarinic receptor antagonists, especially quaternary ammonium muscarinic receptor antagonists such as ipratropium bromide, tiotropium bromide, and oxitropium bromide, as well as the salts, solvates, analogues, congeners, mimetics, stereoisomers, bioisosteres, hydrolysis products, metabolites, precursors, and prodrugs thereof. Xanthine compounds suitable for use as the second drug include, but are not necessarily limited to, theophylline, extended-release theophylline, aminophylline, theobromine, enprofylline, diprophylline, isbufylline, choline theophyllinate, albifylline, arofylline, bamifylline and caffeine, as well as the salts, solvates, analogues, congeners, mimetics, stereoisomers, bioisosteres, hydrolysis products, metabolites, precursors, and prodrugs thereof. Anti-IgE antibodies suitable for use as the second drug include, but are not necessarily limited to, a monoclonal antibody or a genetically engineered antibody that is derived from a monoclonal antibody. Preferably, the anti-IgE antibody is humanized. A particularly preferred humanized anti-IgE antibody is an IgG1 κ monoclonal antibody that specifically binds to human IgE and is marketed under the name of omalizumab. Leukotriene modifiers suitable for use as the second drug include, but are not necessarily limited to, ibudilast, montelukast, pranlukast, and zafirlukast, as well as the salts, solvates, analogues, congeners, mimetics, stereoisomers, bioisosteres, hydrolysis products, metabolites, precursors, and prodrugs thereof. Phosphodiesterase IV inhibitors suitable for use as the second drug include, but are not limited to, roflumilast and cilomilast, as well as the salts, solvates, analogues, congeners, mimetics, stereoisomers, bioisosteres, hydrolysis products, metabolites, precursors, and prodrugs thereof. Phosphodiesterase IV is the predominant isoform in the lung and inhibitors of this enzyme are being considered for the treatment of asthma and COPD.

Yet another embodiment of the invention is a method of treatment of a pulmonary airway disease comprising administering to a subject suffering from pulmonary airway disease:

(1) a controlled-release formulation including an active beta-adrenergic agonist according to the present invention in a therapeutically effective quantity; and

(2) separately, a second drug for treatment of the pulmonary airway disease as desired in a therapeutically effective quantity.

Yet another aspect of the invention is a method of treatment of congestive heart failure comprising administering to a subject suffering from congestive heart failure a therapeutically effective quantity of a controlled release formulation according to the present invention. Again, as described above, the subject can also suffer from a co-morbid disease or condition, in which case the beta-adrenergic inverse agonist in the controlled-release formulation can also be therapeutically effective against the co-morbid condition in the quantity that it is administered to the subject in the formulation, thus again treating two diseases or conditions simultaneously with one drug.

The invention is illustrated by the following Examples. These Examples are included for illustrative purposes only, and are not intended to limit the invention.

### EXAMPLES

### Example 1

### Experiment: Acute Reduction in Airway Function, FEV₁, in Subjects with Mild Asthma with the First 10 mg Dose of Once-Daily Corgard (Nadolol)

Subjects with mild asthma, baseline FEV₁>80% predicted, were enrolled in a clinical study. Subjects went through a two-week washout period and were provided with the inhaler rescue medication Combivent (Salbutamol and ipratropium) for use as needed. However, subjects were advised to not use the rescue medication 8 hours or less before their first dosing visit. On the first dosing visit, 10 mg of Corgard, one half of the 20 mg tablet, was administered orally to each subject. For safety purposes, FEV₁ (forced expiratory volume over 1 second) was monitored prior to drug administration and every 30 minutes for 4 hours in the clinic after the first dose. Peak serum levels of nadolol occur in 3.5 hours, on average, after administration as advised in the Corgard product insert (Monarch Pharmaceuticals, Inc.), consequently, if subjects were to exhibit loss of pulmonary function or even an asthma attack due to the drug, a known risk, then this would most likely happen with the first dose when the change in nadolol drug level is maximal.

If the subjects could tolerate the dose, then subjects were provided with enough drug for a week and advised to return the following week for dose escalation. The study design allowed for 9 study visits for dose escalation, if possible, followed by 3 weeks of treatment at the maximum safely tolerated dose followed by a final visit to monitor pulmonary functions and asthma symptoms.

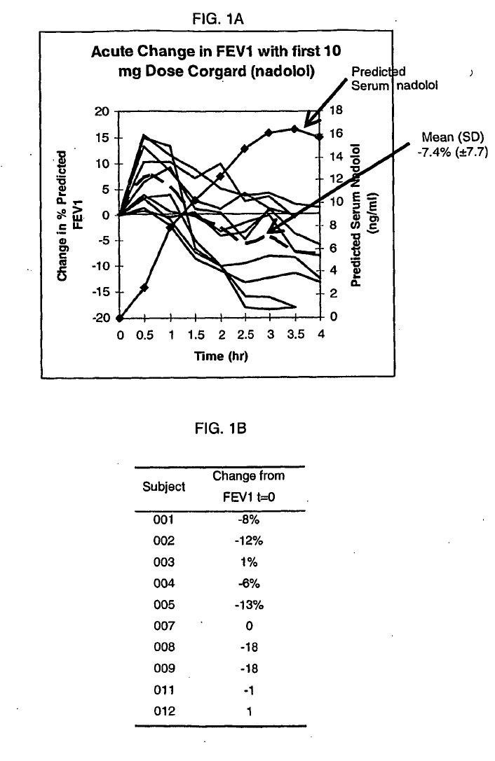

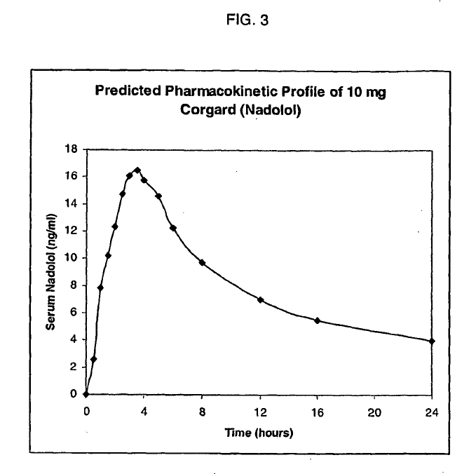

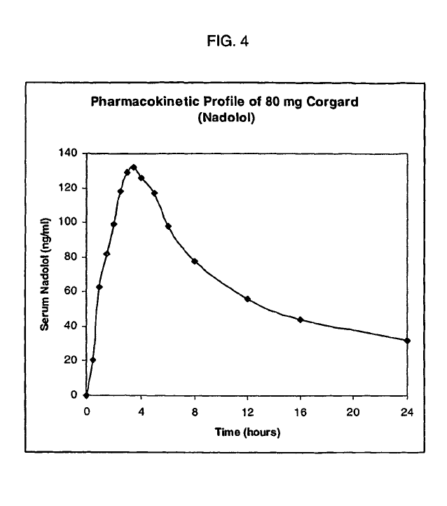

Results: As shown in FIG. 1A most subjects had reductions in their FEV₁ from 30 minutes after dosing to 4 hours, within the time period when peak serum drug levels are expected. For single dosing, Corgard (nadolol) exhibits dose proportionality from 2-80 mg (Dreyfuss et al., Metabolic studies in patients with nadolol: oral and intravenous administration. J Clin Pharmacol. 1977 May-June; 17(5-6):300-7), hence the predicted profile of the 10 mg Corgard (nadolol) pharmacokinetic graph in FIG. 3 have the same pharmacokinetic profile as the experimentally determined 80 mg Corgard (nadolol) graph as shown in FIG. 4. The observed increase in FEV₁ at 30 minutes for the subjects after drug dosing is probably not due to the drug as predicted blood levels would be very low, less than 3 ng/ml. Instead, it is thought that this response may be due to a surge of endogenous epinephrine since this is the first time the subject is receiving an investigational drug in a clinical trial and this surge would be equivalent to a dose of an inhaled beta agonist.

The magnitude change in FEV₁ for all subjects is shown in tabular form in FIG. 1B. The change from baseline FEV₁ at t=0 ranged down to −18%. Since all these asthmatics had a starting FEV₁>80%, even an 18% could still be tolerated by these subjects. However, moderate to severe asthmatics could not tolerate this nearly 20% drop. Predicted FEV₁ is patient-specific and is based on body parameters. For example, a moderate asthmatic typically starts with an FEV₁ 70% of predicted (range 60-80%). If they were administered a 10 mg dose of Corgard and exhibited a similar percentage magnitude drop in FEV₁, they could see their airway FEV₁ drop from 70% to 57% (an 18% drop in FEV₁). FEV₁ of 60% or less of predicted defines patients with severe asthma/pulmonary airway obstruction. For severe asthmatics that start off with FEV₁ of 60% or less of predicted, an 18% drop in FEV₁ could result in a serious airway obstruction requiring emergency intervention.

It would be preferable to avoid clinically meaningful reductions in FEV₁. This would be achievable by the invention having an improved pharmacokinetic profile in which the average Tₘₐₓ of the beta-adrenergic inverse agonist is >4 hours and more preferably >8 hours with a more gradual increase of the drug over the longer time period as shown in Examples 2-11 and their corresponding figures.

Despite the acute airway detriment for most subjects, at the end of the 11 week study, chronic treatment with Corgard (nadolol) resulted in the improvement in airway hyperresponsiveness as measured by PC₂₀ using methacholine in 8 of 10 subjects as shown in FIG. 2A. PC₂₀ methacholine is the provocative dose of methacholine, a muscarinic agonist which causes a minor asthma-like airway constriction, causing a 20% reduction in FEV₁. A very low PC₂₀ means that a subject is very sensitive to low levels of this spasmogen. Consequently, increasing this would result in the subject becoming less sensitive. In FIG. 2B the individual patient PC20 Doubling Doses were graphed relative to drug dose level that they received, either 10, 20 or 40 mg Corgard (nadolol). The dashed line represents 1.5 doubling doses which is considered a clinically meaningful improvement in airway hyperresponsivenss. As can be seen, the two individuals that had a worsening of their airway hyperresponsiveness were on the lowest dose of 10 mg. One patient had an improvement at 10 mg, and this patient only weighed 110 lbs which may account for the improvement. All subjects at both the 20 mg and 40 mg doses had a positive improvement that is consistent with a positive dose response.

Thus, in spite of the fact that most patients exhibited short-term detrimental effects on airway function with the first dose of the drug, most also developed clinically meaningful improvement in their airway hyperresponsiveness.

It is important to note that drug approval from regulatory bodies (e.g. FDA) is based on a risk-benefit assessment. A drug that may be beneficial with long-term treatment is not approvable if the acute detrimental effects place the patient at significant risk thereby underscoring the importance of the improved pharmacokinetic profile of the preferred beta inverse agonist nadolol in this invention.

### Example 2

### Improved Pharmacokinetic Profile for Controlled-Release Nadolol Tₘₐₓ=8 Hours

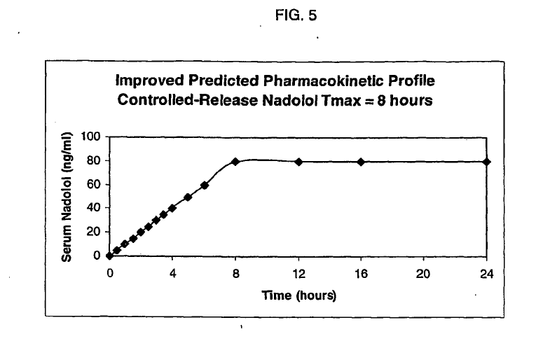

To reduce the potential for reduction in FEV₁ with the first drug dose, an improved pharmacokinetic profile for Nadolol is provided in FIG. 5. For this profile the average Tₘₐₓ is 8 hours and the amount of the drug ascends gradually up to this time point and then is maintained till 24 hours. This is a predicted example, not based on actual results. It is obvious to those skilled in the art that the absolute levels of the drug dose can easily be reduced or increased but still maintain the same shape of the curve over the 24 hour time period.

### Example 3

### Improved Pharmacokinetic Profile for Controlled-Release Nadolol Tₘₐₓ=12 Hours

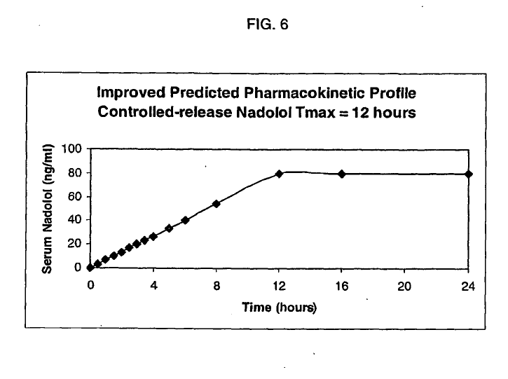

Another improved pharmacokinetic profile is provided in FIG. 6. For this profile the average Tₘₐₓ is 12 hours and the amount of the drug ascends gradually up to this time point and then is maintained till 24 hours. This is a predicted example, not based on actual results. It is obvious to those skilled in the art that the absolute levels of the drug dose can easily be reduced or increased but still maintain the same shape of the curve over the 24 hour time period.

### Example 4

### Improved Pharmacokinetic Profile for Controlled-Release Nadolol Tₘₐₓ=16 Hours

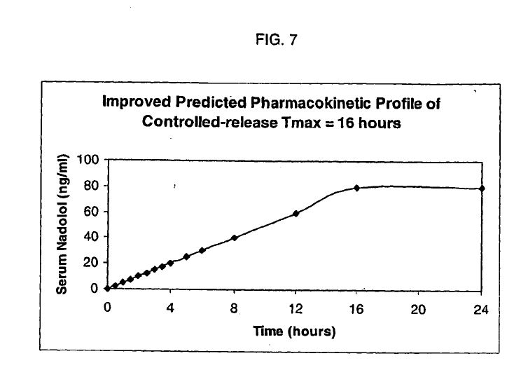

Another improved pharmacokinetic profile is provided in FIG. 7. For this profile the average Tₘₐₓ is 16 hours and the amount of the drug ascends gradually up to 24 hours. This is a predicted example, not based on actual results. It is obvious to those skilled in the art that the absolute levels of the drug dose can easily be reduced or increased but still have the same shape of the curve over the 24 hour time period.

### Example 5

### Improved Pharmacokinetic Profile for Controlled-Release Nadolol Tₘₐₓ=24 Hours

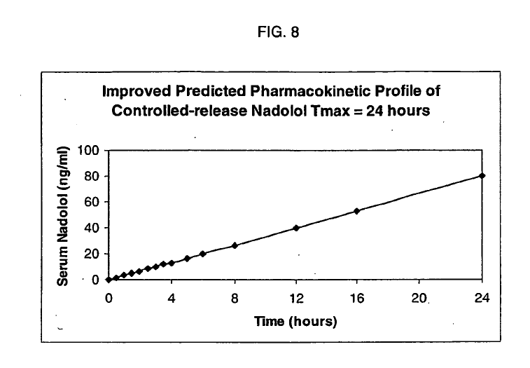

Another improved pharmacokinetic profile is provided in FIG. 8. For this profile the average Tₘₐₓ is 24 hours and the amount of the drug ascends gradually up to this time point. This is a predicted example, not based on actual results. It is obvious to those skilled in the art that the absolute levels of the drug dose can easily be reduced or increased but still maintain the same shape of the curve over the 24 hour time period.

### Example 6

### Improved Pharmacokinetic Profile for Controlled-Release Nadolol for Nocturnal Asthma

Another improved pharmacokinetic profile is provided in FIG. 9 for patients with symptoms of nocturnal asthma between 10 p.m. and 6 a.m. For this profile the average Tₘₐₓ is 8 hours and the amount of the drug ascends gradually up to this time point and then gradually descends for the next 8 hours and then stays flat for the last 8 hours. This is a predicted example, not based on actual results. This pharmacokinetic profile is based on morning dosing typically at 8 a.m. However, for patients that work night shifts, their dosing regime would be correlated with their waking periods and not with the time of day. It is obvious to those skilled in the art that the absolute levels of the drug dose can easily be reduced or increased but still maintain the same shape of the curve over the 24 hour time period.

### Example 7

### Improved Pharmacokinetic Profile #2 for Controlled-Release Nadolol for Nocturnal Asthma

Another improved pharmacokinetic profile is provided in FIG. 10. For this profile the average Tₘₐₓ is 8 hours and the amount of the drug ascends gradually up to this time point and then remains essentially constant in the bloodstream till 16 hours after dosing and then gradually descends for the last 8 hours. This is a predicted example, not based on actual results. This pharmacokinetic profile is for patients that have nocturnal symptoms, particularly around from 2 a.m. to 6 a.m. For patients that work night shifts, their dosing regime would be correlated with their waking periods and not with the time of day. It is obvious to those skilled in the art that the absolute levels of the drug dose can easily be reduced or increased but still maintain the same shape of the curve over the 24 hour time period.

### Example 8

### Improved Pharmacokinetic Enteric-Coated Controlled-Release Nadolol for Asthma

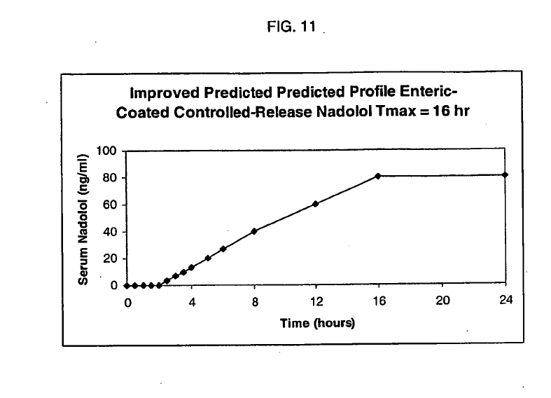

Another improved pharmacokinetic profile is provided in FIG. 11. For this profile, the enteric coating delays any release of the drug until reaches the upper intestinal tract which then dissolves the coating. Subsequently, drug is released from the controlled-release formulation resulting in a gradual increase of drug into the bloodstream until the Tₘₐₓ peak as shown in FIG. 11.

### ADVANTAGES OF THE INVENTION

The present invention provides an improved method of treating chronic pulmonary airway diseases such as asthma, emphysema, and chronic obstructive pulmonary diseases and avoids the tolerance or tachyphylaxis that often is the consequence of conventional therapy with beta-adrenergic agonists. The use of inverse agonists, in essence, forces the body to respond by improving its own signaling mechanisms to counter the pulmonary airway disease. Accordingly, compositions and methods that employ inverse agonists have broad potential for treating such diseases and conditions without the induction of tolerance. This promises superior long-term results in the treatment of such conditions without interfering with short-term acute therapy. The administration of such inverse agonists in a controlled release formulation further provides more effective control of the concentration of the inverse agonist in the blood and the tissues, thereby avoiding exacerbations or other detrimental reactions.

The administration of such inverse agonists in a controlled-release formulation also can be used to treat other co-morbid conditions that exist together with the pulmonary airway disease, including hypertension, angina, congestive heart failure, cardiac arrhythmia, migraines, anxiety, tremor, rosacea, osteoporosis, or other cardiovascular diseases. This provides an effective treatment with two diseases or conditions with one drug, and provides an improved method of treating these co-morbid conditions in patients suffering from pulmonary airway disease or congestive heart failure.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

1. A controlled-release formulation of an active beta-adrenergic inverse agonist comprising:
(a) an active beta-adrenergic inverse agonist in a therapeutically effective quantity; and
(b) at least one agent that controls release of the beta-adrenergic inverse agonist resulting in a pharmacokinetic profile that minimizes acute detrimental reduction in airway function with the first dose and with each successive dose administered to a subject with a condition treatable by the administration of a beta-adrenergic inverse agonist.

2. The controlled-release formulation of claim 1 wherein the average Tₘₐₓ is greater than about 4 hours.

3-4. (canceled)

5. The controlled-release formulation of claim 2 wherein the average Tₘₐₓ is about 16 hours.

6. The controlled-release formulation of claim 2 wherein the average Tₘₐₓ is about 24 hours.

7. The controlled-release formulation of claim 1 wherein the active beta-adrenergic inverse agonist is nadolol and wherein the ratio of Cₘₐₓ/Cₘᵢₙ is about 4.0 or less for once daily dosing.

8. The controlled-release formulation of claim 7 wherein the ratio of Cₘₐₓ/Cₘᵢₙ is about 2.0 or less for once daily dosing.

9. (canceled)

10. The controlled-release formulation of claim 1 wherein the average half-life of the active beta-adrenergic inverse agonist in the blood is >16 hours when the formulation is administered with once-daily dosing.

11. The controlled-release formulation of claim 10 wherein the average half-life of the active beta-adrenergic inverse agonist in the blood is from about 18 hours to about 22 hours when the formulation is administered with once-daily dosing.

12. The controlled-release formulation of claim 10 wherein the average half-life of the active beta-adrenergic inverse agonist in the blood is from about 22 hours to about 28 hours when the formulation is administered with once-daily dosing.

13. The controlled-release formulation of claim 10 wherein the half-life of the active beta-adrenergic inverse agonist in the blood is from about 28 hours to about 38 hours when the formulation is administered with once-daily dosing.

14. The controlled-release formulation of claim 1 wherein the active beta-adrenergic inverse agonist is formulated as part of a formulation selected from the group consisting of: (1) an oral matrix controlled-release formulation; (2) an oral multilayered controlled-release tablet formulation; (3) an oral multiparticulate controlled-release formulation; (4) an oral osmotic controlled-release formulation; (5) an oral chewable controlled-release formulation; and (6) a dermal controlled-release patch formulation.

15-60. (canceled)

61. The controlled-release formulation of claim 1 wherein the active beta-adrenergic inverse agonist has therapeutic activity against a pulmonary airway disease.

62. The controlled-release formulation of claim 61 wherein the active beta-adrenergic inverse agonist has therapeutic activity against a co-morbid condition selected from the group consisting of hypertension, angina, congestive heart failure, cardiac arrhythmia, migraines, anxiety, tremor, rosacea, osteoporosis, and other cardiovascular diseases when administered to a subject that has a pulmonary airway disease together with one or more of these co-morbid conditions.

63. The controlled-release formulation of claim 1 wherein the active beta-adrenergic inverse agonist is selected from the group consisting of nadolol, bupranolol, butoxamine, carazolol, carvedilol, ICI-118,551, levobunolol, metoprolol, propranolol, sotalol, and timolol, and the salts, solvates, analogues, congeners, mimetics, bioisosteres, stereoisomers, hydrolysis products, metabolites, precursors, and prodrugs thereof.

64. The controlled-release formulation of claim 63 wherein the active beta-adrenergic inverse agonist is selected from the group consisting of nadolol, carvedilol, sotalol, metoprolol, timolol, and ICI 118,551, and the salts, solvates, stereoisomers, analogues, congeners, mimetics, bioisosteres, stercoisomers, hydrolysis products, metabolites, precursors, and prodrugs thereof.

65. The controlled-release formulation of claim 64 wherein the active beta-adrenergic inverse agonist is selected from the group consisting of: (a) nadolol; and (b) analogues of nadolol of formula (I) wherein R₁ is hydrogen or lower alkyl, R₂ is hydrogen or lower alkyl, and m and n are 1 to 3, with the proviso that where R₁ and R₂ are both hydrogen and m is 1, n is other than 1.

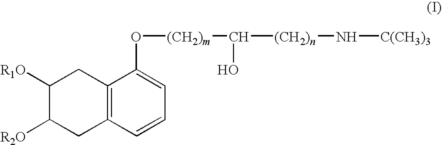

66. The controlled-release formulation of claim 65 wherein the active beta-adrenergic inverse agonist is nadolol.

67-76. (canceled)

77. The controlled-release formulation of claim 1 wherein the controlled-release formulation is formulated for once-daily administration.

78. The controlled-release formulation of claim 77 wherein the active beta-adrenergic inverse agonist is nadolol.

79. The controlled-release formulation of claim 77 wherein the controlled-release formulation is formulated to reduce serum blood levels of the active beta-adrenergic inverse agonist over a time period from about 2 a.m. to about 6 a.m. in order to treat nocturnal asthma.

80-113. (canceled)
